(19) United States
(12) Patent Application Publication
(10) Pub. No.: US 2003/0100031 A1
(43) Pub. Date: May 29, 2003

(54) Integrative assays for monitoring molecular assembly events

(76) Inventors: Steven Dower, Sheffield (GB); Gordon M. Duff, Sheffield (GB)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/167,127

(22) Filed: Jun. 11, 2002

Related U.S. Application Data
(60) Provisional application No. 60/297,305, filed on Jun. 11, 2001.

(51) Int. Cl. G01N 33/53; G01N 33/542; C07K 14/715

(52) U.S. Cl. 435/7.9; 530/350

(57) ABSTRACT

The invention relates to methods, compositions, and apparatus for monitoring molecular assembly events. Monitoring such molecular assembly events, in combination with other assays such as genetic screening, permits the dissection of genetic and non-genetic influences on a particular biological activity.

### CROSS-REFERENCE TO RELATED APPLICATIONS

This application claims priority to U.S. Provisional Application No. 60/297,305, filed Jun. 11, 2001 and incorporated by reference herein in its entirety.

### BACKGROUND

With the completion of the human genome project and the ever-increasing availability of gene sequence information, it has become critically important to understand the relationship between genetic variations and physiological function. Disease states may result from complex interactions between multiple genetic loci and multiple environmental or systemic influences. Although it has become increasingly straightforward to obtain genetic information, the task of characterizing the genetic and non-genetic contributions to a particular disease state remains difficult and time consuming.

The benefits of understanding diseases at a molecular level are potentially enormous. For example, an increasing number of pharmaceutical agents are known to act on particular protein targets, and in the future, the molecular activities of most pharmaceuticals will be understood. In theory, this knowledge will permit the tailoring of a patient's therapeutic regimen so as to correct the molecular basis of a disease state. However, tailored drug therapy can only be achieved if the molecular basis of disease is accurately assessed. For example, inflammatory responses are an important component of many disease states. The transcription factor NF-κB mediates many inflammatory events in response to signals received from the interleukin-1 (IL-1) and tumor necrosis factor alpha (TNFα) signaling pathways (as well as several other pathways). If a patient is suffering from a disorder caused by excessive activation of NF-κB, it would benefit the physician to understand whether to select a therapeutic that inhibits the TNFα signaling pathway, one that targets the IL-1 pathway, or neither. A system for determining the activation state of individual signal transduction pathways would permit a physician to characterize the molecular basis of many illnesses and prescribe appropriate medication.

The molecular basis for a physiological state may be understood through an analysis of protein-protein interactions. Many cellular functions are regulated through the controlled assembly and disassembly of molecular complexes. For example, activation of essentially any signal transduction pathway causes changes in the interactions between the various protein components of that pathway. Receptor tyrosine kinases, upon activation, stimulate the formation of large complexes of phosphotyrosine binding proteins that mediate downstream signaling events. The activity of the NF-κB transcription factor is, in part, regulated by its interaction with the inhibitor protein IκB. In addition, many metabolic events are thought to be carried out by complexes of proteins. For example, DNA replication is now understood to be performed by a large and dynamic complex of polymerases and accessory proteins.

It is an object of this invention to provide compositions, methods and apparatus for monitoring molecular assembly events that integrate the environmental, biochemical and genetic influences acting on a particular biological system. Such compositions, methods and apparatus will have many uses in areas including, but not limited to, linking genetic variations to molecular and physiological events, drug screening, diagnostics, therapy selection and dosing, patient monitoring and environmental safety.

### BRIEF SUMMARY OF THE INVENTION

In certain aspects, the invention provides methods, compositions, and apparatus for monitoring one or more molecular assembly events. In certain embodiments, such a molecular assembly event has the following properties: (1) the assembly event acts as a surrogate measure for a biological activity (for example, but not limited to, a signal transduction pathway or metabolic pathway), (2) the assembly event integrates both genetic and non-genetic effects on the biological activity; and (3) the assembly event is specific to the biological activity of interest, minimizing cross-talk with other biological activities that are not of interest.

One aspect of the present application relates to interactive sensor pairs. In certain embodiments, interactive sensor pairs comprise a first polypeptide and a second polypeptide, where the first polypeptide is stably attached to a first reactive module, and the second polypeptide is stably attached to a second reactive module. When the first polypeptide and second polypeptide are present in a complex, the first and second reactive modules interact so as to produce an output signal that is quantitatively or qualitatively different from the output signal that is produced when the first and second polypeptides are not in a complex. By monitoring the output signal, it is possible to determine whether the first and second polypeptides have formed a complex. In other embodiments, the invention relates to detection reagents comprising an interactive sensor pair, and cells and membranes comprising interactive sensor pairs. In preferred embodiments, an interactive sensor pair measures the activation state of a receptor, preferably a multiple-ligand-responsive receptor. In further embodiments, either the first or second polypeptide is stably attached to a reactive module, so long as the formation of a complex between the first and second polypeptides modulates the output signal of the reactive module.

In other aspects, the invention provides methods for measuring the ability of a sample to modulate a molecular assembly event. Such methods comprise placing a sample in contact with a detection reagent comprising an interactive sensor pair and measuring the output signal. A change in the output signal is an indication that the sample modulates the molecular assembly event. In other embodiments, the sample may comprise a test substance. The change in output signal resulting from contacting the test substance with the interactive sensor pair may be used to detect a test substance that acts as an agonist or antagonist of a molecular assembly event.

In another aspect, the invention provides a method for measuring the change in a molecular assembly event in response to a test condition. A method of the invention may comprise measuring the output signal produced by an interactive sensor pair in a control condition and exposing the interactive sensor pair to a test condition. The change in output signal from the control condition to the test condition indicates the change in assembly state in response to the test condition.

In a further aspect, the invention provides methods for integrating genetic and non-genetic information. In certain embodiments the invention provides methods for determining the effect of an allelic pattern on a biological activity in a subject. Such methods may include detecting an allelic pattern in a nucleic acid sample obtained from a subject; contacting a biological sample obtained from the subject with a detection reagent; and measuring the output signal, wherein the output signal integrates the effects of said allelic pattern on the biological activity in the subject. In preferred embodiments, the detection reagent comprises an interactive sensor pair having a first polypeptide stably attached to a first reactive module and a second polypeptide stably attached to a second reactive module, wherein said first polypeptide comprises a multiple-ligand-responsive receptor, and wherein said second polypeptide comprises a protein that binds to said first polypeptide when said first polypeptide is activated by ligand.

In a further aspect, the invention provides methods for generating database systems for integrating genetic and non-genetic information. Information for generating database systems may be obtained by detecting an allelic pattern in nucleic acid samples obtained from a plurality of subjects; contacting biological samples obtained from said plurality of subjects with a detection reagent, wherein said detection reagent comprises an interactive sensor pair; measuring the output signals produced by the interactive sensor pair in response to each biological sample; and/or obtaining clinical status information from said plurality of subjects. An entry for each type of information is entered into the database system. Preferably, each type of record is linked to the other types of record. The invention further provides computer systems comprising a database system generated according the methods described herein. For example, a computer system of the invention may comprise a database system containing, for each subject, linked records reflecting genotype, output signal and clinical status, and a user interface allowing a user to selectively view information regarding allelic patterns and output signals.

In yet other aspects, the invention provides methods for selecting an appropriate targeted therapeutic for a subject, comprising detecting an allelic pattern in a nucleic acid sample obtained from said subject; contacting a biological sample obtained from said subject with a detection reagent, wherein said detection reagent comprises an interactive sensor pair; and measuring the output signal. Preferably, the interactive sensor pair, as monitored through the output signal, integrates the effects of said allelic pattern on said biological activity in said subject. In general a targeted therapeutic is selected to compensate for abnormal biological activity that may be reflected by the output signal, and preferably the targeted therapeutic compensates for abnormal biological activity that is caused, in part, by the subject's genotype.

In certain embodiments, a method for selecting an appropriate target therapeutic for a subject is a computer-assisted method. Such a method may comprise contacting a biological sample obtained from a subject with a detection reagent, wherein said detection reagent comprises an interactive sensor pair, and measuring the output signal. The output signal may then be compared against a database comprising output signal information from a plurality of subjects and further comprising clinical status information from a plurality of subjects. It is contemplated that one may use a computer interface to identify in the database any clinical conditions correlated with the level of biological activity reflected in the output signal. Accordingly, one may select a targeted therapeutic to ameliorate or prevent the correlated condition. In certain embodiments, such a method may be used to predict the onset of conditions before such conditions are evident by other clinical criteria.

In yet a further aspect, the invention provides apparatus for measuring the ability of a sample to modulate a molecular assembly event. In certain embodiments, an apparatus may comprise a sample chamber for receiving a sample, a detection reagent comprising an interactive sensor pair, a mechanism for contacting the detection reagent with the sample, and a sensor capable of measuring the output signal of said sensor pair. The output signal as measured by the sensor indicates the degree to which the sample modulates the molecular assembly state.

In another embodiment, the invention provides an apparatus for providing appropriate dosing of a therapeutic that modulates a molecular assembly event. In some variations, the apparatus comprises a sample chamber for receiving a sample from a patient, a detection reagent comprising an interactive sensor pair, a mechanism for contacting the detection reagent with the sample, a sensor capable of measuring the output signal of the sensor pair, and a dosing element for providing an appropriate dose of the therapeutic in response to the output signal. In certain embodiments, if the output signal indicates an unheathfully high level of a biological activity represented by the molecular assembly event, then the dosing element provides an appropriate dose of a therapeutic that inhibits the biological activity. In other embodiments, if the output signal indicates an unheathfully low level of a biological activity then the dosing element provides an appropriate dose of a therapeutic that increases the activity.

In further embodiments, the invention relates to fusion proteins comprising a polypeptide fused to a reactive module polypeptide, and optionally fused to a polypeptide to facilitate protein purification. Exemplary polypeptides that facilitate protein purification include histidine tags (purification by nickel or other metal affinity), glutathione-S-transferase (purification by affinity for glutathione), cellulose binding domains (purification by affinity for cellulose), etc. The further relates to nucleic acids encoding such fusion proteins, expression vectors and cells comprising the nucleic acids.

In certain embodiments, the invention relates to kits comprising a detection reagent, and optionally comprising additional components, such as a device for obtaining a sample from a subject (e.g. a syringe, a capillary stick, a biopsy implement, etc.), instructions, an output signal reader, etc.

### BRIEF DESCRIPTION OF THE DRAWINGS

FIG. 1: Genomic nucleotide sequence for the IL-1A gene (SEQ ID NO:1).

FIG. 2: Amino acid sequence for the IL-1α protein (SEQ ID NO:2).

FIG. 3: Genomic nucleotide sequence for the IL-1B gene (SEQ ID NO:3).

FIG. 4: Amino acid sequence for the IL-1β protein (SEQ ID NO:4).

FIG. 5: Genomic nucleotide sequence for the IL-1RN gene (SEQ ID NO:5).

FIG. 6: Amino acid sequence for the IL-1Ra protein (SEQ ID NO:6).

FIG. 7: Nucleotide sequence for the IL-1RN gene encoding the intracellular form (SEQ ID NO:7).

FIG. 8: Amino acid sequence for the intracellular form of the IL-1Rac protein (SEQ ID NO:8).

FIG. 9: Nucleotide sequence for the IL-1Rac gene (SEQ ID NO:9).

FIG. 10: Amino acid sequence for the IL-1Rac protein (SEQ ID NO:10).

FIG. 11: Nucleotide sequence for the IL-1R1 gene (SEQ ID NO:11).

FIG. 12: Amino acid sequence for the IL-1R1 protein (SEQ ID NO:12).

FIG. 13: Amino acid sequence for an IL-1R1-CFP truncation fusion protein (SEQ ID NO:13).

FIG. 14: Amino acid sequence for an IL-1Rac-YFP truncation fusion protein (SEQ ID NO:14).

FIG. 15: Amino acid sequence for an IL-1R1-CFP full length fusion protein (SEQ ID NO:15).

FIG. 16: Amino acid sequence for an IL-1Rac-CFP full length fusion protein (SEQ ID NO:16).

FIG. 17: Diagram of FRET using truncation fusion constructs.

FIG. 18: Diagram of FRET using full length fusion constructs.

FIG. 19: Fluorescence images of HEK293 cells expressing full-length fusion constructs.

FIG. 20: Fluorescence images of HEK293 cells expressing full-length fusion constructs.

FIG. 21: IL-1β induces an increase in FRET (truncation constructs).

### DETAILED DESCRIPTION OF THE INVENTION

Definitions:

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

An “active portion” of a polypeptide that is used in a detection reagent is a portion that retains the ability to bind in a regulated manner to another polypeptide of the detection reagent and, if applicable, retains the ability to bind to one or more ligands.

The term “allele” refers to the different sequence variants found at different polymorphic regions. For example, an allele from the IL-1 region, IL-1RN (VNTR), has at least five different alleles. The sequence variants may be single or multiple base changes, including without limitation insertions, deletions, or substitutions, or may be a variable number of sequence repeats.

The term “allelic pattern” refers to the identity of an allele or alleles at one or more polymorphic regions in the genetic material of an organism. For example, an allelic pattern may consist of a single allele at a polymorphic site, as for IL-1RN (VNTR) allele 1, which is an allelic pattern having at least one copy of IL-1RN allele 1 at the VNTR of the IL-1RN gene loci. Alternatively, an allelic pattern may consist of either a homozygous or heterozygous state at a single polymorphic site. For example, IL1-RN (VNTR) allele 2,2 is an allelic pattern in which there are two copies of the second allele at the VNTR marker of IL-1RN and that corresponds to the homozygous IL-RN (VNTR) allele 2 state. Alternatively, an allelic pattern may consist of the identity of alleles at more than one polymorphic site. Although the examples above are from the IL-1 region, an allelic pattern may refer to a polymorphic site in any part of a genome.

The term “cell” includes not only to a particular subject cell, but to the progeny or potential progeny of such a cell. Because certain modifications may occur in succeeding generations due to either mutation or environmental influences, such progeny may not, in fact be identical to the parent cell, but are still included within the scope of the term as used herein. A “recombinant cell” is a cell that comprises a recombinant nucleic acid construct. The terms “comprise” and “comprising” is used in the inclusive, open sense, meaning that additional elements may be included.

The term “cytokine” is used to refer to small proteins or biological factors (in the range of 5-20 kD) that are released by cells and have specific effects on cell-cell interaction, communication and behavior of other cells. In particular, “cytokine” refers to interleukins, lymphokines and several related signaling molecules such as TNF and interferons. Exemplary cytokines are IL-1, IL-2, IL-6, IL-8, IL-10, IL-13, IL-18, TNFα and interferon γ.

A “detection reagent” is a composition comprising an interactive sensor pair that may be brought into contact with a sample. The interactive sensor pair may be free in solution, or one or both members of the interactive sensor pair may, for example, be adhered to a substrate, incorporated into a material such as a gel, lipid bilayer or micelle, or expressed in a cell. The detection reagent may include, for example, a solid or semi-solid substrate such as beads, plates, fibers, sheets, gels (e.g. polyacrylamide, agarose) or any other substrate that permits adherence or incorporation of a member of the interactive sensor pair. The detection reagent may be a solution of soluble and insoluble components. The reagent may, for example, include cells expressing the interactive sensor pair. The cells may be adhered to a substrate or suspended in solution. As a further example, the reagent may include hydrophobic membranes with a member of the interactive sensor pair inserted therein.

A “disorder associated allele” or “an allele associated with a disorder” refers to an allele whose presence in a subject indicates that the subject has or is susceptible to developing a particular disorder.

A “fusion protein” or “fusion polypeptide” refers to a chimeric protein as that term is known in the art and may be constructed using methods known in the art. Many exemplary fusion proteins are a polypeptide chain comprising two or more amino acid sequences that do not regularly occur together in a single polypeptide chain. Optionally, each of the two or more amino acid sequences provides a distinct property or biochemical activity. In various embodiments, the fusion polypeptide may comprise one or more amino acid sequences linked to a first polypeptide. In the case where more than one amino acid sequence is fused to a first polypeptide, the fusion sequences may be multiple copies of the same sequence, or alternatively, may be different amino acid sequences. The fusion polypeptides may be fused to the N-terminus, the C-terminus, or the N- and C-terminus of the first polypeptide. Exemplary fusion proteins include polypeptides comprising a fluorescent polypeptide such as a GFP, glutathione S-transferase,tag (GST-tag), histidine tag (His-tag), an immunoglobulin domain or an immunoglobulin binding domain. A “fusion construct” is a nucleic acid encoding a fusion protein. A “tranlational fusion” means that two coding regions are in frame with no functional intervening stop codons, allowing translation as a single fusion polypeptide.

A “haplotype” refers to a set of alleles that are inherited together as a group (are in linkage disequilibrium). As used herein, haplotype is defined to include those haplotypes that occur at statistically significant levels (pcorr <0.05). For example, “an IL-1 haplotype” refers to a haplotype in the IL-1 loci. At least two IL-1 proinflammatory haplotypes are known. The IL-1 (44112332) haplotype is associated with decreased IL-receptor antagonist activity, whereas the IL-1 (33441461) haplotype is associated with increased IL-1 α and β agonist activity. The IL-1 (44112332) haplotype includes the following alleles: IL-1RN (+2018) allele 2; IL-1RN (VNTR) allele 2; IL-1A (222/223) allele 4; IL-1A (gz5/gz6) allele 4; IL-1A (−889) allele 1; IL-1B (+3954) allele 1; IL-1B (−511) allele 2; gaat.p33330 allele 3; Y31 allele 3; IL-1RN exon 1ic (1812) allele 2; IL-1RN exon 1ic (1868) allele 2; IL-1RN exon 1ic (1887) allele 2; Pic (1731) allele 2; IL-1A (+4845) allele 1; IL-1B (+6912) allele 1; IL-1B (−31) allele 2. The IL-1 (33441461) haplotype includes the following alleles: IL-1RN (+2018) allele 1; IL-1RN (VNTR) allele 1; IL-1A (222/223) allele 3; IL-1A (gz5/gz6) allele 3; IL-1A (−889) allele 2; IL-1B (+3954) allele 2; IL-1B (−511) allele 1; gaat.p33330 allele 4; Y31 allele 6; IL-1RN exon 1ic (1812) allele 1; IL-1RN exon 1ic (1868) allele 1; IL-1RN exon 1ic (1887) allele 1; Pic (1731) allele 1; IL-1A (+4845) allele 2; IL-1B (+6912) allele 2; IL-1B (−31) allele 1.

An “IL-1R-like receptor” is a single-pass transmembrane receptor with at least about 20-30% amino acid sequence identity to the human IL-1RI and mediates a response to an extracellular ligand. Heterodimerization is an important part of activation of IL-1R1-like receptors. IL-1R1 is an example of an IL-1R1-like receptor, as are any forms of IL-1R1 modified by changing the amino acid sequence without substantially altering the ligand and accessory binding protein functions of the IL-1R1 receptor (e.g. conservative amino acid sequence changes, suitable truncations, etc.)

“IL-1X (Z) allele Y “refers to a particular allelic form, designated Y, occurring at an IL-1 locus polymorphic site in gene X, wherein X is IL-1A, B, or RN or some other gene of the IL-1 gene loci, and positioned at or near nucleotide Z, wherein nucleotide Z is numbered relative to the major transcriptional start site, which is nucleotide +1, of the particular IL-1 gene X. As further used herein, the term “IL-1X allele (Z)” refers to all alleles of an IL-1 polymorphic site in gene X positioned at or near nucleotide Z. For example, the term “IL-1RN (+2018) allele” refers to alternative forms of the IL-1RN gene at marker +2018. “IL-1RN (+2018) allele 1” refers to a form of the IL-1RN gene which contains a cytosine (C) at position +2018 of the sense strand. Clay et al., Hum. Genet. 97:723-26, 1996. “IL-1RN (+2018) allele 2” refers to a form of the IL-1RN gene which contains a thymine (T) at position +2018 of the plus strand. When a subject has two identical IL-1RN alleles, the subject is said to be homozygous, or to have the homozygous state. When a subject has two different IL-1RN alleles, the subject is said to be heterozygous, or to have the heterozygous state. The term “IL-1RN (+2018) allele 2,2” refers to the homozygous IL-1 RN (+2018) allele 2 state. Conversely, the term “IL-1RN (+2018) allele 1,1” refers to the homozygous IL-1 RN (+2018) allele 1 state. The term “IL-1RN (+2018) allele 1,2” refers to the heterozygous allele 1 and 2 state. Similar nomenclature may be used with alleles of other loci, such as IL-18R (Z) allele Y.

“Increased risk” or “increased susceptibility” refers to a statistically higher frequency of occurrence of the disease or condition in an individual carrying a particular polymorphic allele in comparison to the frequency of occurrence of the disease or condition in a member of a population that does not carry the particular polymorphic allele.

An “infection” includes any viral, fungal, bacterial or parasitic infection.

The term “including” is used herein to mean “including but not limited to”. “Including” and “including but not limited to” are used interchangeably.

An “interactive sensor pair” or “sensor pair” includes a first polypeptide and a second polypeptide, where the first polypeptide is stably attached to a first reactive module, and the second polypeptide is stably attached to a second reactive module. When the first polypeptide and second polypeptide become bound together in a complex, the first and second reactive modules interact so as to produce a quantitatively or qualitatively different output signal. By monitoring this output signal, it is possible to determine whether the first and second polypeptides have formed a complex. An alternate form of an interactive sensor pair is one in which the first polypeptide is stably attached to one or more reactive modules so as to produce a quantitatively or qualitatively different output signal when in a complex with a second polypeptide. The second polypeptide need not be stably attached to a reactive module (or, in other words, the second polypeptide, through its effects on the first polypeptide and the attached reactive module(s) acts as a reactive module itself). A reactive module need not be stably attached to one end or the other of a polypeptide, but may be attached to a central portion of a polypeptide or may even interrupt the linear sequence of the polypeptide, so long as it does not disrupt the ability of the polypeptide to interact with its partner.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject IL-1 polypeptides preferably includes no more than 10 kilobases (kb) of nucleic acid sequence which naturally immediately flanks the IL-1 gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

A “lipid layer” includes any of the various structures formed by a plurality of lipids when contacted with a substantially hydrophilic medium (e.g. an aqueous medium). For example, lipids in an aqueous medium tend to form micelles, lipid bilayers, vesicles, lipid monolayers, sandwiches, coiled sheets, etc., with the general principle being that typically hydrophobic portions of the lipids are shielded from the aqueous medium and the hydrophilic portions are in contact with the aqueous medium. Lipids include triglycerides, fatty acids, phospholipids (e.g. phosphatidylcholine, phosphatidylserine, phosphatidylinositol), waxes, esters, sterols, glycolipids, etc. Lipid layers may include mixtures of lipids and may also include substantial portions of other materials, such as proteins and sugars.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. As used herein, the term “linkage disequilibrium” also refers to linked sequences. Alleles that co-occur at expected frequencies are said to be in “linkage equilibrium” or “not linked.” When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern. An example of linkage disequilibrium is that which occurs between the alleles at the IL-1RN (+2018) and IL-1RN (VNTR) polymorphic sites. The two alleles at IL-1RN (+2018) are >97% in linkage disequilibrium with the two most frequent alleles of IL-1RN (VNTR), which are allele 1 and allele 2.

The term “molecular assembly event”, “assembly event” or “molecular assembly state” is intended to refer to an interaction, or a change in an interaction, between specific biological molecules, including but not limited to nucleic acids, lipids, proteins and carbohydrates. A molecular assembly event may refer to, for example, the formation of a complex, the dissolution of a complex, or a change in the components of a complex. For example, the association of a receptor and an accessory protein is an assembly event, as is the multimerization of an actin filament, the interaction between a G-protein coupled receptor and a G-protein, etc.

“Multiple-ligand-responsive receptor” is a receptor that modulates a signaling pathway in response to more than one natural ligand (a ligand that naturally occurs in the same subject organism as the receptor). For example, the IL-1 receptor responds to IL-1α, IL-1β and IL-1ra.

The term “or” as used herein should be understood to mean “and/or”. The term “propensity” as used herein in reference to a condition or disease state, as in “propensity” for a condition or disease, is used interchangeably with the expressions “susceptibility” or “predisposition”. The term “propensity” as used in reference to a condition or disease state indicates that an individual is at increased risk for the future development of a condition or disease. For example, if an allele or a molecular assembly event is discovered to be associated with or predictive of a particular disease or condition, an individual carrying the allele has a greater propensity for developing the particular disease or condition. By “partially purified”, with respect to protein preparations, it is meant that the proteins have been previously separated from other cellular or viral proteins. For instance, in contrast to whole cell lysates, the proteins of reconstituted conjugation system, together with the substrate protein, can be present in the mixture to at least 50% purity relative to all other proteins in the mixture, more preferably are present at least 75% purity, and even more preferably are present at 90-95% purity.

The term “purified” refers to an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 80 percent of all species present in the composition, more than about 85%, 90%, 95%, 99% or more of all species present. The object species may be purified to essential homogeneity (contaminant species cannot be detected in the composition by conventional detection methods) wherein the composition consists essentially of a single species. A skilled artisan may purify a polypeptide of the invention using standard techniques for protein purification in light of the teachings herein. Purity of a polypeptide may be determined by a number of methods known to those of skill in the art, including for example, amino-terminal amino acid sequence analysis, gel electrophoresis and mass-spectrometry analysis.

“Reactive module” is used herein to indicate any molecule or composite that undergoes a measurable change in an output signal when it is brought into close proximity with a second reactive module or that causes a measurable change in an output signal of a second reactive module when brought in close proximity (without necessarily producing any output signal itself). The first and second reactive modules may be the same or different, so long as one or more output signal changes when they come into close proximity with each other. Exemplary reactive modules include polypeptide reactive modules such as fluorescent proteins, enzymes, etc.

The term “recombinant nucleic acid construct” includes any combination of nucleic acid sequences that was generated by a technique of molecular biology. For example, recombinant nucleic acids may include nucleic acid sequences fused together by ligation, by polymerase chain reaction, by integration of a nucleic acid into a chromosome or episome of a cell (e.g. by transposon, by homologous recombination, by non-homologous recombination, by phage insertion, etc.). A “recombinant nucleic acid expression construct” includes any recombinant nucleic acid construct that comprises an expressible nucleic acid and appropriate cis-acting sequences to permit expression of the expressible nucleic acid (e.g. a promoter, an enhancer, etc.). An exemplary recombinant nucleic acid expression construct is a plasmid carrying a gene operably linked to an IPTG-inducible promoter. Another exemplary recombinant nucleic acid expression construct is an enhancer inserted into the genome next to the endogenous gene encoding the desired protein. An “exogenously regulated expression construct” is a recombinant nucleic acid expression construct wherein the rate of generation, degradation or accumulation of expressed nucleic acid is at least partially controlled by an external factor (i.e. “inducer”) that may be readily provided by one of skill in the art. An exogenously regulated expression construct is generally designed to be at least partially controlled by one or more specific inducers. For example, a Plac promoter is regulated by exogenously supplied galactose or variants of galactose such as isothiopyranogalactoside (IPTG). Promoters regulated by temperature sensitive transcription factors are induced by changes in temperature. Pxyl promoters are induced with xylose, and many other exogenously regulated expression constructs, along with the appropriate inducers, are known in the art.

A “sample” incudes material obtained from a subject or an object of interest. For example, samples may be obtained from a human or animal subject, a plant, a cell culture or an environmental location, such as a water or air sample. The sample also includes materials that have been processed or mixed with other materials. For example, a blood sample may be processed to obtain serum, red blood cells, etc., each of which may be considered a sample.

A “stable attachment” as used in reference to a polypeptide and a reactive module includes any interaction that withstands the assay conditions used for detecting an output signal. Exemplary stable attachments include covalent bonds, such as peptide bonds, and non-covalent interactions, such as hydrogen bonds, hydrophobic interactions, salt bridges, etc. A stable attachment may also comprise multiple connecting bonds of multiple types between the reactive module and the subject polypeptide. “Substantially enrich”, as used herein, means to enrich by at least 10%, more preferably by at least 30%, and still more preferably at least about 50%, at least one component of the whole cell lysate compared to another component of the whole cell lysate. The term “semi-purified cell extract” is also intended to include the lysate from a cell, when the cell has been treated so as to have substantially more, or substantially less, of a given component than a control cell. For example, a cell which has been modified (by, e.g., recombinant DNA techniques) to produce none (or very little) of a particular cellular component, will, upon cell lysis, yield a semi-purified cell extract.

A “targeted therapeutic” is a therapeutic composition that modulates a specific molecular mechanism related to a disease state. A targeted therapeutic can comprise any type of compound, including, for example, a protein, peptide, peptidomimetic, small molecule, nucleic acid, or nutraceutical.

A “test substance” can comprise essentially any element, chemical compound (including a nucleic acid, protein, peptide, carbohydrate or lipid) or mixture thereof, including a nutraceutical or small molecule drug.

“Toll-like receptors” are transmembrane molecules comprising multiple extracellular leucine-rich repeats, a single transmembrane domain and an intracellular signaling domain termed the TIR domain (Toll and IL-1 receptor related). TIR domains are generally 20-30% identical across the family and fold to form a central parallel beta-sheet of about five beta-strands and surrounded by about five alpha-helices on both sides of the sheet (Xu et al. (2000) Nature 408: 111-115).

“Transcriptional regulatory sequence” is a generic term used throughout the specification to refer to DNA sequences, such as initiation signals, enhancers, and promoters, which induce or control transcription of protein coding sequences with which they are operably linked.

A “transgenic animal” refers to any non-human animal, preferably a non-human mammal, bird or an amphibian, in which one or more of the cells of the animal contain a heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of an IL-1 polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques. The term is intended to include all progeny generations. Thus, the founder animal and all F1, F2, F3, and so on, progeny thereof are included.

The term “vector” refers to a nucleic acid molecule, which is capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”.

Interactive Sensor Pairs

In certain aspects the invention provides interactive sensor pairs. An interactive sensor pair comprises a first polypeptide and a second polypeptide, where, in certain embodiments, the first polypeptide is stably attached to a first reactive module, and the second polypeptide is stably attached to a second reactive module. When the first polypeptide and second polypeptide become bound together in a complex, the first and second reactive modules interact so as to produce a quantitatively or qualitatively different output signal. By monitoring this output signal, it is possible to determine whether the first and second polypeptides have formed a complex. In other embodiments, the first polypeptide is stably attached to a reactive module, and the output signal differs when the first polypeptide is bound in a complex with a second polypeptide. In certain embodiments, the first polypeptide comprises two reactive modules that produce an altered output signal when the first polypeptide is bound in a complex with a second polypeptide.

First and second reactive modules of the invention may be essentially any pair of compounds that undergo a change in one or more output signal when they come into close proximity. An output signal may be any property that is measurable, including, but not limited to, fluorescence excitation spectrum, fluorescence emission spectrum, quantum efficiency, enzymatic activity, absorption spectrum or ligand binding affinity. Output signals may increase or decrease when the first and second polypeptides interact, so long as the interaction produces a measurable change. It is preferable that the reactive modules have minimal natural tendency to associate with each other because such association could drive the interaction between the first and second polypeptides.

In preferred embodiments, reactive modules comprise fluorescent proteins, for example fluorescent proteins isolated from jellyfish, corals and other coelenterates. Exemplary fluorescent proteins include the many variants of the green fluorescent protein (GFP) of Aequoria victoria. Variants may be brighter, dimmer, or have different excitation and/or emission spectra. Certain variants are altered such that they no longer appear green, and may appear blue, cyan, yellow, red (termed BFP, CFP, YFP and RFP, respectively) or have other emission spectra. Reactive modules comprising fluorescent proteins may be stably attached to polypeptides through a variety of covalent and noncovalent linkages, including, for example, peptide bonds (e.g. expression as a fusion protein), chemical cross-linking and biotin-streptavidin coupling. For examples of fluorescent proteins, see U.S. Pat. Nos. 5,625,048; 5,777,079; 6,066,476; 6,124,128; Prasher et al. (1992) Gene, 111:229-233; Heim et al. (1994) Proc. Natl. Acad. Sci., USA, 91:12501-04; Ward et al. (1982) Photochem. Photobiol., 35:803-808 ; Levine et al. (1982) Comp. Biochem. Physiol., 72B:77-85; Tersikh et al. (2000) Science 290: 1585-88.

In other embodiments, reactive modules of the invention may comprise polypeptides that have been modified with a fluorescent moiety. Fluorescent moieties are well known in the art and include derivatives of fluorescein, benzoxadioazole, coumarin, eosin, Lucifer Yellow, pyridyloxazole and rhodamine. These and many other exemplary fluorescent moieties may be found in the Handbook of Fluorescent Probes and Research Chemicals (2000, Molecular Probes, Inc.), along with methodologies for modifying polypeptides with such moieties. Exemplary proteins that fluoresce when combined with a fluorescent moiety include, yellow fluorescent protein from Vibrio fischeri (Baldwin et al. (1990) Biochemistry 29:5509-15), peridinin-chlorophyll a binding protein from the dinoflagellate Symbiodinium sp. (Morris et al. (1994) Plant Molecular Biology 24:673:77) and phycobiliproteins from marine cyanobacteria such as Synechococcus, e.g., phycoerythrin and phycocyanin (Wilbanks et al. (1993) J. Biol. Chem. 268:1226-35). These proteins require flavins, peridinin-chlorophyll a and various phycobilins, respectively, as fluorescent co-factors.

In particularly preferred embodiments, the output signal of fluorescent or fluorescently-labeled reactive modules changes when the modules come into close proximity as a result of Fluorescence Resonance Energy Transfer (FRET). Fluorescent molecules having the proper emission and excitation spectra that are brought into close proximity with one another can exhibit FRET. The fluorescent molecules are chosen such that the emission spectrum of one of the molecules (the donor molecule) overlaps with the excitation spectrum of the other molecule (the acceptor molecule). The donor molecule is excited by light of appropriate intensity within the donor's excitation spectrum. The donor then emits the absorbed energy as fluorescent light. The fluorescent energy it produces is quenched by the acceptor molecule. FRET can be manifested as a reduction in the intensity of the fluorescent signal from the donor, reduction in the lifetime of its excited state, and/or re-emission of fluorescent light at the longer wavelengths (lower energies) characteristic of the acceptor. When the fluorescent proteins physically separate, FRET effects are diminished or eliminated. (U.S. Pat. No. 5,981,200).

For example, a cyan fluorescent protein is excited by light at roughly 425-450 nm wavelength and emits light in the range of 450-500 nm. Yellow fluorescent protein is excited by light at roughly 500-525 nm and emits light at 525-500 nm. If these two proteins are placed in solution, the cyan and yellow fluorescence may be separately visualized. However, if these two proteins are forced into close proximity with each other, the fluorescent properties will be altered by FRET. The bluish light emitted by CFP will be absorbed by YFP and re-emitted as yellow light. This means that when the proteins are stimulated with light at wavelength 450 nm, the cyan emitted light is greatly reduced and the yellow light, which is not normally stimulated at this wavelength, is greatly increased. FRET is typically monitored by measuring the spectrum of emitted light in response to stimulation with light in the excitation range of the donor and calculating a ratio between the donor-emitted light and the acceptor-emitted light. When the donor:acceptor emission ratio is high, FRET is not occurring and the two fluorescent proteins are not in close proximity. When the donor: acceptor emission ratio is low, FRET is occurring and the two fluorescent proteins are in close proximity. In this manner, the interaction between a first and second polypeptide fused to a first and second reactive module, wherein the first and second reactive modules are donor and acceptor fluorescent molecules, respectively, may be measured.

The occurrence of FRET also causes the fluorescence lifetime of the donor fluorescent moiety to decrease. This change in fluorescence lifetime can be measured using a technique termed fluorescence lifetime imaging technology (FLIM) (Verveer et al. (2000) Science 290: 1567-1570; Squire et al. (1999) J. Microsc. 193: 36; Verveer et al. (2000) Biophys. J. 78: 2127). Global analysis techniques for analyzing FLIM data have been developed. These algorithms use the understanding that the donor fluorescent moiety exists in only a limited number of states each with a distinct fluorescence lifetime. Quantitative maps of each state can be generated on a pixel-by-pixel basis.

In a further embodiment, the first and second reactive modules provide a Bioluminescence Resonance Energy Transfer System (BRET). In BRET, one reactive module is an enzyme that produces (or destroys) a fluorescent product (or substrate) and another reactive module is a fluorescent protein that undergoes resonant energy transfer with the fluorescent product (or substrate). In one embodiment, a BRET system comprises a luciferase from Renilla and the fluorescent protein is GFP. Exemplary BRET methodologies are described in Kroeger et al., J Biol Chem. 2001 Apr 20;276(16):12736-43 and Xu et al., Proc Natl Acad Sci USA. 1999 January 5;96(1):151-6.

In an additional embodiment, the first and second reactive modules comprise portions of a holoenzyme that, when brought into close proximity, permit (or inhibit) an enzyme activity, and wherein the enzyme activity yields (or destroys) a detectable product (or substrate). For example, an exemplary system employs the bacterial enzyme, beta-galactosidase, the gene product of LacZ. The functional beta-galactosidase enzyme is a tetramer consisting of 4 identical subunits. Several domains from each polypeptide chain are involved in the formation of this tetrameric enzyme. A mutant form exists that is generally inactive, but restoration of the enzymatic activity occurs when the mutant combines with a second mutant form of beta-galactosidase to provide the structure necessary to form an intact beta-galactosidase complex. This phenomenon is called intra-cistronic complementation or alpha-complementation. The enzyme activity that results from beta-galactosidase complementation is a direct measurement of the protein-protein interaction.

In certain embodiments, the first polypeptide of the interactive sensor pair is a transmembrane receptor. The second polypeptide is preferably a protein that associates with the first polypeptide specifically when the receptor is activated. The interaction between the first and second polypeptides is, preferably, a specific marker for activation of the transmembrane receptor. In particularly preferred embodiments, the second polypeptide is a second transmembrane protein that does not bind directly to the ligand of the receptor and may be considered an accessory protein.

In yet another embodiment, the first polypeptide is a multiple-ligand-responsive receptor, and the interaction between the first polypeptide and the second polypeptide is a measure of the total activation state of the first polypeptide in response to one or more ligands. Preferably, the multiple-ligand-responsive receptor is responsive to more than one or more than two naturally occurring ligands.

In certain embodiments, the interactive sensor pair will be expressed in a cell. In view of this specification, many variations can be designed. For example, one member of the sensor pair may be expressed in a cell, while the other member is produced and supplied exogenously. In another variation, the first and second polypeptides of the interactive sensor pair interact best when the two polypeptides are placed on separate cells. Any transfectable cell may be used to express one or both members of an interactive sensor pair. In certain embodiments, the interactive sensor pair is used as expressed in one or more cell types, and in further embodiments, one or more components of an interactive sensor pair is partially purified (or greater purification) from from a cell expressing the component from a recombinant nucleic acid construct, and the cell is used as a production agent. A list of exemplary cells, not intended to be exhaustive or limiting, includes HEK293T cells, Chinese Hamster Ovary cells, HeLa cells, fibroblasts, keratinocytes, insect cells (e.g., Drosophila S2 cells, Sf9 cells) yeast cells, immune cell-derived cell lines, etc.

In yet other embodiments, one or both members of the interactive sensor pair may be placed in a cell-free environment. Such an environment could be a membrane fraction isolated from cells, or an artificial membrane constructed in vitro. Membrane proteins may also be solubilized with detergent. Depending on the detergent and the concentration used, the detergent/protein mixture may or may not form micelles. In general, a detergent will form micelles when it is present at a concentration above the so-called critical concentration. The detergent Triton X-100, which has a low critical concentration, is commonly used to form micelles with transmembrane proteins. The detergent octyl glucoside, which has a high critical concentration, is often used to solubilize proteins without forming micelles. For each protein, the specific detergent and/or membrane compositions that best retain desired biological activities must be determined experimentally. Such optimization is, in view of this specification, well within the capabilities of one skilled in the art. A variety of methods for using detergents as well as additional exemplary detergents may be found in: Guide to Protein Purification: Methods in Enzymology (Methods in Enzymology Series, Vol. 182) by Murray P. Deutscher(Editor), et al (spiral bound, July 1997).

Members of the interactive sensor pair may be present in the same or different micelles or membranes. For example, the first member of the interactive sensor pair may be prepared in a population of liposomes, and the second member of the interactive pair may be prepared in a second population of liposomes. These two populations may then be mixed. If desired, the liposomes can then be induced to fuse.

While essentially any polypeptides may be used in interactive sensor pairs, preferred polypeptides include receptors and their accessing proteins. Examples of such preferred polypeptides include, without limitation, the IL-1 receptor and its accessory protein (IL-1R1 and IL-1Rac), IL-18 receptor and its accessory protein (IL-18R and IL-18Rac), and mammalian Toll-like receptor 1 and Toll-like receptor 2 (TLR1 and TLR2).

Interleukin-1 Receptor

IL-1α and IL-1β proteins are pro-inflammatory cytokines involved in regulating many local and systemic responses of the immune and inflammatory systems. IL-1α and IL-1β affect cells primarily by interacting with transmembrane receptors that transduce the signal across the plasma membrane and initiate an intracellular signal transduction cascade. IL-1α and IL-1β have very similar biological activities and interact with the same receptors. Collectively, IL-1α and IL-1β are referred to as IL-1.

The primary receptor for these factors is the type I IL-1 receptor (IL-1RI). The active signaling complex consists of the IL-1 ligand (IL-1α or IL-1β), the type I receptor and the IL-1 receptor accessory protein (IL-1Rac). A type II receptor (IL-1RII), as well as soluble forms of the type I and type II receptors appear to act as decoy receptors to compete for bioavailable IL-1. In addition, a natural inhibitor of IL-1 signaling, IL-1 receptor antagonist (IL-1ra), is produced by monocytes. IL-1ra is also produced by hepatocytes and is a major component of the acute phase proteins produced in the liver and secreted into the circulation to regulate immune and inflammatory responses. IL-1Rac and IL-1RI interact to form the activated signaling complex when IL-1RI is bound to IL-1α or IL-1β but not IL-1ra. Furthermore, IL-1Rac is essential for IL-1 signaling (Wesche et al. (1997) J. Biol. Chem. 12: 7727-7731; Greenfelder et al. (1995) J. Biol. Chem. 23: 13757-13765). The formation of the IL-1Rac-IL-1RI complex is a highly specific indicator of activation of the IL-1RI receptor.

An exemplary interactive sensor pair of the invention comprises a first polypeptide comprising a portion of IL-1RI sufficient to form a complex with IL-1Rac upon binding IL-1α, β or another activating ligand. The exemplary interactive sensor pair further comprises a second polypeptide comprising a portion of IL-1Rac sufficient to bind to IL-1RI when IL-1RI is bound to IL-1α, β or another activating ligand.

The active IL-1 signaling complex activates several intracellular signal transduction pathways, including the activities of NF-κB and AP-1 described above. In signaling, IL-1 influences the activity of a host of factors including: PI-3 kinase, phospholipase A2, protein kinase C, the JNK pathway, 5-lipoxygenase, cyclooxygenase 2, p38 MAP kinase, p42/44 MAP kinase, p54 MAP kinase, Rac, Ras, TRAF-6, TRAF-2 and many others. IL-1 also affects expression of a large number of genes including: members of the IL-1 gene cluster, TNF, other interleukin genes (2, 3, 6, 8, 12, 2R, 3R and 5R), TGF-β, fibrinogen, matrix metalloprotease 1, collagen, elastase, leukemia inhibiting factor, IFN α, β, γ, COX-2, inducible nitric oxide synthase, metallothioneins, and many more.

The IL-1 polypeptides, IL-1α and IL-1β, are abundantly produced by activated macrophages that have been stimulated with bacterial lipopolysaccharide (LPS), TNF, IL-1 itself, other macrophage-derived cytokines, or contact with CD4+T cells. The IL-1 promoter contains several regulatory elements including a cAMP responsive element, an AP-1 binding site and an NF-κB binding site. Both NF-κB and AP-1 (Jun and Fos) must be activated and translocated to the nucleus in order to regulate transcription. NF-κB is normally retained in the cytoplasm through binding with IκB. The NF-κB -IκB complex is disrupted by phosphorylation of IκB. IκB phosphorylation can be regulated by signaling from cell-surface receptors via activation of mitogen-activated protein kinase (MAP kinse) pathways and other kinase pathways. Jun and Fos are also substrates for regulatory kinases, such as JNK, in the case of Jun.

The IL-1A and B transcripts are translated into pro-proteins by a process that may also be regulated by MAP kinase pathways. Inhibitors of MAP kinase phosphorylation such as trebufelone decrease translation of IL-1 transcripts. The IL-1 α and β precursor proteins require myristoylation for localization to the membrane and conversion to mature IL-1 by the Interleukin Converting Enzyme (ICE). Other extracellular proteases may also play a minor role in IL-1 maturation, including trypsin, elastase, chymotrypsin and mast cell chymase. ICE can be inhibited by several agents including the εICE isoform, antibodies to the ICE α, β and γ isoforms, the cow pox-produced Crm-A protein and an endogenous tetrapeptide competitive inhibitor.

Interleukin-18 Receptor

IL-18, or IGIF (interferon-gamma inducing factor), is an IL-1-related, pro-inflammatory cytokine, which regulates systemic and local inflammation. Mature IL-18 is roughly 18 kilodaltons and, among other activities, stimulates the production of interferon-gamma by T cells (Okamura et al. (1995) Infect. Immun. 63: 3966; Gu et al. (1997) Science 275:206-209).

The IL-18 receptor system closely resembles the IL-1 system. IL-18 binds to a receptor, the IL-18 receptor (IL-18R, also known as the IL-1R related protein) (Parnet et al. (1996) J. Biol. Chem. 271: 3967-3970). IL-18 binds its receptor at both high and low affinity sites. IL-18 selectively binds IL-18R. Much like IL-1RI, an accessory protein is necessary for IL-18R activity. The accessory protein is termed IL-18ac (also known as IL-1Rac-like protein). Complex formation of IL-18R and IL-18ac is likely to be a highly specific indicator of IL-18 binding (Debets et al. (2000) J. Immunol. 165:4950-6).

IL-18 may also have an IL-1ra equivalent termed IL-1H. The protein sequence of IL-1H is mostly related to IL-1ra with a similarity of 36%. A short form of IL-1H was identified, lacking a 40-amino acid segment in the amino-terminal region of the protein. IL-1H binds the IL-18 receptor, but not the IL-1 receptor, therefore IL-1H may be a ligand for the IL-18 receptor and may play a role in regulating the IL-18 signaling pathway (Pan et al. (2001) Cytokine 13:1-7).

Toll-like Receptors

The innate immune system utilizes a set of receptors to detect the presence of pathogens and mount a variety of host defense mechanisms such as phagocytosis, complement activation and expression of pro-inflammatory genes. In Drosophila, the Toll receptor functions both in early development and in the immune response. A wide range of Toll-like receptors (TLRs) also function in innate immunity in vertebrates.

The precise ligands for most TLRs are still unknown. Innate immunity is generally directed at conserved microbial structures that are relatively conserved across a related group of microorganisms. It is generally thought that TLRs are involved in the recognition of these conserved structures. Studies have shown that responsiveness to lipopolysaccharide (LPS) relies on TLR4, while peptidoglycan recognition depends on TLR2. Both of these TLRs are probably responsive to a range of microbial compounds.

Recent evidence suggests that agonist-driven heterodimerization may be critical for activation of TLR signaling pathways. TLR1 modulates the signaling activity and apparent ligand specificity of TLR2, suggesting that these receptors heterodimerize upon ligand binding to initiate the signaling cascade. In other words, for certain ligands, signaling may depend upon both TLR1 and TLR2. (Wyllie et al. (2000) J. Immunol. 165: 7125-7132; Sato et al. (2000) J. Immunol. 165: 7096-7101; Medzhitov et al. (2000) Trends Microbiol. 8: 452-456).

Detection Methods

In certain aspects the invention provides methods for determining the effect of a sample or condition on a molecular assembly event. Methods for determining the ability of a sample to modulate a molecular assembly event are also provided. In preferred embodiments, the molecular assembly event is a surrogate for the activation state of a receptor.

The output signal from interactive sensor pairs may be used to measure the effect of a sample on a molecular assembly event. A sample may be essentially any material of interest, including but not limited to samples from living subjects or environmental samples, and including solid, fluid or gaseous samples. Samples may be prepared by a variety of means such as, but not limited to: liquifying, solubilizing, sonicating, suspending in liquid, purifying, or centrifuging. Samples may also be used in an essentially unaltered state. The sample is contacted with the desired interactive sensor pair and the change in the output signal is measured. In certain embodiments the change in the output signal represents the effect of the sample on the interactive sensor pair, and by extension, the activation state of the molecular assembly event represented by the interactive sensor pair. For example, a blood sample may contain an IL-1α, IL-1β and IL-1ra. Each of these components contributes differently to the activation of the IL-1 receptor. The IL-1RI activating capability of the blood may be measured using an interactive sensor pair comprising an active portion of IL-1R1 as the first polypeptide and comprising an active portion of IL-1Rac as the second polypeptide. In an exemplary scenario where the output signal to be measured is FRET and the association of the sensor pair is indicative of receptor activation, then an increase in FRET will indicate that the receptor is in a more activated state. A receptor agonist would increase FRET, while a receptor antagonist would decrease FRET. A mixture of agonists and antagonists would produce a measurable net effect that indicates the tendency of the mixture as a whole to activate, deactivate or have no effect on the receptor activation state.

It is understood that interactive sensor pairs may be used to screen for and identify novel agonists and antagonists or other molecules that modulate a biological activity. In one embodiment a single screening assay may comprise contacting an interactive sensor pair with a test compound and measuring the signal output. In an exemplary embodiment, cells or tissue that produce ligand are contacted with a test compound in the presence of an interactive sensor pair. The output signal of the exemplary sensor pair reflects receptor activation, which in turn reflects ligand production. In this manner, the effects of a test compound on ligand production may be determined, and compounds that affect ligand production, stability or processing may be identified. High throughput screening assays could also be devised taking advantage of multiwell plates, plate readers, microarrays of proteins and other such technologies that could, in view of this specification, be appropriately selected and implemented by one of skill in the art. Methods may be adapted to select agents from, for example, nutraceutical, chemical, pharmaceutical and biological libraries that have desired effects on various receptor systems.

In general it will be desirable to establish a baseline signal for an interactive sensor pair and then to compare this baseline against a test condition. The change in output signal from the baseline condition to the test condition indicates the effect of the test condition on activation state of the subject receptor. It is also possible to measure output signal without reference to a baseline, particularly if many measurements are being performed and can be compared against each other.

It is further understood that interactive sensor pairs may be placed in an in vivo or ex vivo milieu to detect the tendency of that milieu to modulate the sensor pair. Interactive sensor pairs may be implanted within an organism and monitored externally, or the appropriate monitoring technology may also be implanted along with technology for recording and/or transmitting data. Interactive sensor pairs may also be placed in an environmental setting, including, without limitation, a body of water, sewage system, soil, air, exhaust pipe or any other setting in which it would be desirable to measure the tendency of that milieu to modulate the activity of a receptor.

Interactive sensor pairs may be used to determine the effects on the desired molecular assembly event of a wide range of factors including, without limitation, drug candidates; nutraceuticals; biological molecules; infective agents and their products; vaccines; toxins; work-place pollutants, allergens; other xenobiotics, different forms of radiation; physiological stresses; hormones; life-style changes.

Integrative Methods and Databases

In certain aspects, the invention provides methods of integrating genetic and non-genetic influences on a biological activity. For example, essentially any of the methods, apparatus and/or compositions related to interactive sensor pairs described herein may be combined with information about the genotype of the subject or sample material. Such integrative methods are useful, for example, for relating genetic variations to molecular processes, and may, if desired, be further related to phenotypes, such as the clinical condition of a subject.

In certain embodiments the invention provides methods for determining the effect of an allelic pattern on a biological activity in a subject. Such methods include detecting an allelic pattern in a nucleic acid sample obtained from a subject; contacting a biological sample obtained from the subject with a detection reagent; and measuring the output signal, wherein the output signal integrates the effects of said allelic pattern on the biological activity in the subject. Further information and benefit may be derived by the comparison in a plurality of subjects the relationship between an allelic pattern and a biological activity. In general, it is preferable to detect the presence of many different alleles at multiple loci. In this manner it is possible to identify particular alleles that are best associated with a certain biological activity. General methods for genetic testing are described in greater detail below.

In a further aspect, the invention provides methods for generating database systems for integrating genetic and non-genetic information. Information for generating database systems may be obtained by detecting an allelic pattern in nucleic acid samples obtained from a plurality of subjects; contacting biological samples obtained from said plurality of subjects with a detection reagent, wherein said detection reagent comprises an interactive sensor pair; measuring the output signals produced by the interactive sensor pair in response to each biological sample; and/or obtaining clinical status information from said plurality of subjects. An entry for each type of information is entered into the database system. An entry may be an exact data output, or it may be a processed form of the data. For example, the primary output signal may be a quantitative fluorescence measurement, but this primary signal may be converted to a different scale, such as a simple numerical scale designed to reflect the level of activity. Likewise, genotype or clinical status information may be distilled, simplified, augmented or in any other way transformed for the purpose of becoming a database entry. Preferably, each type of entry is linked to the other types of entry on a subject-by-subject basis. In view of this specification, suitable database systems for the storage and interconnection of data entries will be apparent to those of skill in the art.

In an exemplary embodiment, presented here for the purposes of illustration and not intended to be limiting, the method may be directed to IL-1 genotypes and biological activities. An IL-1 allelic pattern is tested in patients. Such allelic patterns may comprise one or more alleles and exemplary allelic patterns are described below. Samples, such as blood samples are obtained from the patients and contacted with a detection reagent comprising an interactive sensor pair that is a surrogate for IL-1 activity. Such a surrogate may, for example, be an interactive sensor pair comprising IL-1R1 as a first polypeptide and IL-1rac as a second polypeptide. The output signal is measured and represents a measure of the IL-1R1-affecting activities (eg. IL-1α, β, receptor antagonist, IL-1R type II, etc.) present in each sample. The comparison of output signal and genotype permits one of skill in the art to draw conclusions about the effect of certain IL-1 allelic patterns on IL-1R1-affecting activities.

The invention further provides computer systems comprising a database system generated according the methods described herein. For example, a computer system of the invention may comprise a database system containing, for each subject, linked records reflecting genotype, output signal and clinical status, and a user interface allowing a user to selectively view information regarding allelic patterns and output signals.

In yet other aspects, the invention provides methods for selecting an appropriate targeted therapeutic for a subject, comprising detecting an allelic pattern in a nucleic acid sample obtained from said subject; contacting a biological sample obtained from said subject with a detection reagent, wherein said detection reagent comprises an interactive sensor pair; and measuring the output signal. Preferably, the interactive sensor pair, as monitored through the output signal, integrates the effects of said allelic pattern on said biological activity in said subject. In general a targeted therapeutic is selected to compensate for abnormal biological activity that may be reflected by the output signal, and preferably the targeted therapeutic compensates for abnormal biological activity that is caused, in part, by the subject's genotype.

In certain embodiments, a method for selecting an appropriate target therapeutic for a subject is a computer-assisted method. Such a method may comprise contacting a biological sample obtained from a subject with a detection reagent, wherein said detection reagent comprises an interactive sensor pair, and measuring the output signal. The output signal may then be compared against a database comprising output signal information from a plurality of subjects and further comprising clinical status information from a plurality of subjects. It is contemplated that one may use a computer interface to identify in the database any clinical conditions correlated with the level of biological activity reflected in the output signal. Accordingly, one may select a targeted therapeutic to ameliorate or prevent the correlated condition. In certain embodiments, such a method may be used to predict the onset of conditions before such conditions are evident by other clinical criteria.

Genetic Analysis

With the development of simple and inexpensive genetic screening methodology, it is now possible to identify polymorphisms that indicate a propensity to develop disease, even when the disease is of polygenic origin. The number of diseases that can be screened by molecular biological methods continues to grow with increased understanding of the genetic basis of multifactorial disorders.

Genetic screening (also called genotyping or molecular screening), can be broadly defined as testing to determine if a patient has mutations (or alleles or polymorphisms) that either cause or alter a disease state or are “linked” to the mutation causing or altering a disease state. Linkage refers to the phenomenon that DNA sequences which are close together in the genome have a tendency to be inherited together. Two sequences may be linked because of some selective advantage of co-inheritance. More typically, however, two polymorphic sequences are co-inherited because of the relative infrequency with which meiotic recombination events occur within the region between the two polymorphisms. The co-inherited polymorphic alleles are said to be in linkage disequilibrium with one another because, in a given human population, they tend to either both occur together or else not occur at all in any particular member of the population. Indeed, where multiple polymorphisms in a given chromosomal region are found to be in linkage disequilibrium with one another, they define a quasi-stable genetic “haplotype.” In contrast, recombination events occurring between two polymorphic loci cause them to become separated onto distinct homologous chromosomes. If meiotic recombination between two physically linked polymorphisms occurs frequently enough, the two polymorphisms will appear to segregate independently and are said to be in linkage equilibrium.

While the frequency of meiotic recombination between two markers is generally proportional to the physical distance between them on the chromosome, the occurrence of “hot spots” as well as regions of repressed chromosomal recombination can result in discrepancies between the physical and recombinational distance between two markers. Thus, in certain chromosomal regions, multiple polymorphic loci spanning a broad chromosomal domain may be in linkage disequilibrium with one another, and thereby define a broad-spanning genetic haplotype. Furthermore, where a disease-causing mutation is found within or in linkage with this haplotype, one or more polymorphic alleles of the haplotype can be used as a diagnostic or prognostic indicator of the likelihood of developing the disease. This association between otherwise benign polymorphisms and a disease-causing polymorphism occurs if the disease mutation arose in the recent past, so that sufficient time has not elapsed for equilibrium to be achieved through recombination events. Therefore identification of a human haplotype which spans or is linked to a disease-causing mutational change, serves as a predictive measure of an individual's likelihood of having inherited that disease-causing mutation. Importantly, such prognostic or diagnostic procedures can be utilized without necessitating the identification and isolation of the actual disease-causing lesion. This is significant because the precise determination of the molecular defect involved in a disease process can be difficult and laborious, especially in the case of multifactorial diseases such as inflammatory disorders.

Indeed, the statistical correlation between a disorder and an IL-1 polymorphism does not necessarily indicate that the polymorphism directly causes the disorder. Rather the correlated polymorphism may be a benign allelic variant which is linked to (i.e. in linkage disequilibrium with) a disorder-causing mutation which has occurred in the recent human evolutionary past, so that sufficient time has not elapsed for equilibrium to be achieved through recombination events in the intervening chromosomal segment. Thus, for the purposes of diagnostic and prognostic assays for a particular disease, detection of a polymorphic allele associated with that disease can be utilized without consideration of whether the polymorphism is directly involved in the etiology of the disease. Furthermore, where a given benign polymorphic locus is in linkage disequilibrium with an apparent disease-causing polymorphic locus, still other polymorphic loci which are in linkage disequilibrium with the benign polymorphic locus are also likely to be in linkage disequilibrium with the disease-causing polymorphic locus. Thus these other polymorphic loci will also be prognostic or diagnostic of the likelihood of having inherited the disease-causing polymorphic locus. Indeed, a broad-spanning human haplotype (describing the typical pattern of co-inheritance of alleles of a set of linked polymorphic markers) can be targeted for diagnostic purposes once an association has been drawn between a particular disease or condition and a corresponding human haplotype. Thus, the determination of an individual's likelihood for developing a particular disease of condition can be made by characterizing one or more disease-associated polymorphic alleles (or even one or more disease-associated haplotypes) without necessarily determining or characterizing the causative genetic variation.

Many methods are available for detecting specific alleles at human polymorphic loci. The preferred method for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in an individual. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA TM is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated; polymorphisms that occur in runs of the same nucleotide can result in signals that are proportional to the length of the run (Syvanen, A.-C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the methods described herein. In a preferred embodiment, a nucleic acid sample is obtained from a bodily fluid, e.g., blood, obtained by known techniques (e.g. venipuncture) or saliva. Alternatively, nucleic acid tests can be performed on dry samples (e.g. hair or skin). When using RNA or protein, the cells or tissues that may be utilized must express an IL-1 gene.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele of an IL-1 proinflammatory haplotype and having about 5, 10, 20, 25, or 30 nucleotides around the mutation or polymorphic region. In a preferred embodiment of the invention, several probes capable of hybridizing specifically to desired allelic variants are attached to a solid phase support, e.g., a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-Beta Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, a method of detecting a polymorphism includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of an IL-1 proinflammatory haplotype under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, the allele of an IL-1 proinflammatory haplotype is identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety of sequencing reactions known in the art can be used to directly sequence the allele. Exemplary sequencing reactions include those based on techniques developed by Maxim and Gilbert ((1977) Proc. Natl Acad Sci USA 74:560) or Sanger (Sanger et al (1977) Proc. Nat. Acad. Sci USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; and Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of an IL-1 locus haplotype is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify an allele. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163; Saiki et al (1989) Proc. Natl Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labeled target DNA.

Alternatively, allele specific amplification technology which depends on selective PCR amplification may -be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g,. biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Another embodiment of the invention is directed to kits for detecting alleles. This kit may contain one or more oligonucleotides, including 5′ and 3′ oligonucleotides that hybridize 5′ and 3′ to at least one allele. In preferred embodiments, the allele is an allele of an IL-1 locus haplotype. PCR amplification oligonucleotides should hybridize between 25 and 2500 base pairs apart, preferably between about 100 and about 500 bases apart, in order to produce a PCR product of convenient size for subsequent analysis.

Particularly preferred primer pairs for use in the diagnostic method of the invention include the following:

(SEQ ID No. 1)

5′ ATG GTT TTA GAA ATC ATC AAG CCT AGG GCA 3′ and

(SEQ ID No. 2)

5′ AAT GAA AGG AGG GGA GGA TGA CAG AAA TGT 3′;

(SEQ ID No. 3)

5′ TGG CAT TGA TCT GGT TCA TC-3′ and

(SEQ ID No. 4)

5′ GTT TAG GAA TCT TCC CAC TT-3′;

(SEQ ID No. 5)

5′ CTC AGG TGT CCT CGA AGA AAT CAA A 3′ and

(SEQ ID No. 6)

5′ GCT TTT TTG CTG TGA GTC CCG 3′;

(SEQ ID NO. 7)

5′-CTC.AGC.AAC.ACT.CCT.AT-3′ and

(SEQ ID NO. 8)

5′-TCC.TGG.TCT.GCA.GCT.AA-3′;

(SEQ ID NO. 9)

5′-CTA TCT GAG GAA CAA ACT AGT AGC-3′ and

(SEQ ID NO. 10)

5′-TAG GAC ATT GCA CCT AGG GTT TGT -3′;

(SEQ. ID No. 11)

5′ ATT TTT TTA TAA ATC ATC AAG CCT AGG GCA 3′ and

(SEQ. ID No. 12)

5′ AAT TAA AGG AGG GAA GAA TGA CAG AAA TGT 3′;

(SEQ ID NO. 13)

5′-AAG CTT GTT CTA CCA CCT GAA CTA GGC.-3′ and

(SEQ ID NO. 14)

5′-TTA CAT ATG AGC CTT CCA TG.-3′.

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

The kit may, optionally, also include DNA sampling means. DNA sampling means are well known to one of skill in the art and can include, but not be limited to substrates, such as filter papers, the AmpliCard™ (University of Sheffield, Sheffield, England S10 2JF; Tarlow, J W, et al., J. of Invest. Dermatol. 103:387-389 (1994)) and the like; DNA purification reagents such as Nucleon™ kits, lysis buffers, proteinase solutions and the like; PCR reagents, such as 10× reaction buffers, thermostable polymerase, dNTPs, and the like; and allele detection means such as the HinfI restriction enzyme, allele specific oligonucleotides, degenerate oligonucleotide primers for nested PCR from dried blood.

Genetics of the IL-1 Gene Cluster

The IL-1 gene cluster is on the long arm of chromosome 2 (2q13) and contains at least the genes for IL-1α (IL-1A), IL-1β (IL-1B), and the IL-1 receptor antagonist (IL-1RN), within a region of 430 Kb (Nicklin, et al. (1994) Genomics, 19: 382-4). The agonist molecules, IL-1α and IL-1β, have potent pro-inflammatory activity and are at the head of many inflammatory cascades. Their actions, often via the induction of other cytokines such as IL-6 and IL-8, lead to activation and recruitment of leukocytes into damaged tissue, local production of vasoactive agents, fever response in the brain and hepatic acute phase response. All three IL-1 molecules bind to type I and to type II IL-1 receptors, but only the type I receptor transduces a signal to the interior of the cell. In contrast, the type II receptor is shed from the cell membrane and acts as a decoy receptor. The receptor antagonist and the type II receptor, therefore, are both anti-inflammatory in their actions.

Certain alleles from the IL-1 gene cluster are already known to be associated with particular disease states. For example, IL-1RN allele 2 has been shown to be associated with coronary artery disease (PCT/US/98/04725, and U.S. Ser. No. 08/813456), osteoporosis (U.S. Pat. No. 5,698,399), nephropathy in diabetes mellitus (Blakemore, et al. (1996) Hum. Genet. 97(3): 369-74), alopecia areata (Cork, et al., (1995) J. Invest. Dermatol. 104(5 Supp.): 15S-16S; Cork et al. (1996) Dermatol Clin 14: 671-8), Graves disease (Blakemore, et al. (1995) J. Clin. Endocrinol. 80(1): 111-5), systemic lupus erythematosus (Blakemore, et al. (1994) Arthritis Rheum. 37: 1380-85), lichen sclerosis (Clay, et al. (1994) Hum. Genet. 94: 407-10), and ulcerative colitis (Mansfield, et al. (1994) Gastoenterol. 106(3): 637-42).

In addition, the IL-1A allele 2 from marker −889 and IL-1B (TaqI) allele 2 from marker +3954 have been found to be associated with periodontal disease (U.S. Pat. No. 5,686,246; Kornman and diGiovine (1998) Ann Periodont 3: 327-38; Hart and Kornman (1997) Periodontol 2000 14: 202-15; Newman (1997) Compend Contin Educ Dent 18: 881-4; Kornman et al. (1997) J. Clin Periodontol 24: 72-77). The IL-1A allele 2 from marker −889 has also been found to be associated with juvenile chronic arthritis, particularly chronic iridocyclitis (McDowell, et al. (1995) Arthritis Rheum. 38: 221-28). The IL-1B (TaqI) allele 2 from marker +3954 of IL-1B has also been found to be associated with psoriasis and insulin dependent diabetes in DR3/4 patients (di Giovine, et al. (1995) Cytokine 7: 606; Pociot, et al. (1992) Eur J. Clin. Invest. 22: 396-402). Additionally, the IL-1RN (VNTR) allele 1 has been found to be associated with diabetic retinopathy (see U.S. Ser. No. 09/037472, and PCT/GB97/02790). Furthermore allele 2 of IL-1RN (VNTR) has been found to be associated with ulcerative colitis in Caucasian populations from North America and Europe (Mansfield, J. et al., (1994) Gastroenterology 106: 637-42). Interestingly, this association is particularly strong within populations of ethnically related Ashkenazi Jews (PCT WO97/25445).

These IL-1 locus polymorphisms represent single base variations within the IL-1A/IL-1B/IL-1RN gene cluster. The IL-1A (+4845) polymorphism is a single base variation (allele 1 is G, allele 2 is T) at position +4845 within Exon V of the IL-1A gene which encodes the inflammatory cytokine IL-1a (Gubler, et al.(1989) Interleukin, inflammation and disease (Bomford and Henderson, eds.) p.31-45, Elsevier publishers; and Van den velden and Reitsma (1993) Hum Mol Genetics 2:1753-50). The IL-1A (+4845) polymorphism occurs in the coding region of the gene and results in a single amino acid variation in the encoded protein (Van den Velden and Reitsma (1993) Hum Mol Genet 2: 1753). The IL-1B (+3954) polymorphism was first described as a Taq I restriction fragment length polymorphism (RFLP) (Pociot et al. (1992) Eur J Clin Invest 22: 396-402) and has subsequently been characterized as a single base variation (allele 1 is C, allele 2 is T) at position +3954 in Exon V of the IL-1B gene (di Giovine et al. (1995) Cytokine 7: 600-606). This single nucleotide change in the open reading frame of IL-1B does not appear to qualitatively affect the sequence of the encoded IL-1 beta polypeptide because it occurs at the third position of a TTC phenylalanine codon (F) of allele 1 and therefore allele 2 merely substitutes a TTT phenylalanine codon at this position which encodes amino acid 105 of the IL-1B gene product. In addition, the IL-1RN (+2018) polymorphism (Clay et al. (1996) Hum Genet 97: 723-26) is a single base variation (allele 1 is T, allele 2 is C), also referred to as exon 2 (8006) (GenBank:X64532 at 8006). Finally, the IL-RN variable number of tandem repeats (VNTR) polymorphism occurs within the second intron the IL-1 receptor antagonist encoding gene (Steinkasserer (1991) Nucleic Acids Res 19: 5090-5). Allele 2 of the of the IL-1RN (VNTR) polymorphism corresponds to two repeats of an 86-base pair sequence, while allele 1 corresponds to four repeats, allele 3 to three repeats, allele 4 to five repeats, and allele 5 to six repeats (Tarlow et al. (1993) Hum Genet 91: 403-4).

The following alleles of the IL-1 (33221461) haplotype are in linkage disequilibrium, and therefore, any allele that is linkage disequilibrium with one of the following may also be presumed to a part of the haplotype and in linkage disequilibrium with the others of that haplotype:

allele 3 of the 222/223 marker of IL-1A
allele 3 of the gz5/gz6 marker of IL-1A
allele 2 of the −889 marker of IL-1A
allele 2 of the +3954 marker of IL-1B
allele 1 of the −511 marker of IL-1B
allele 4 of the gaat.p33330 marker
allele 6 of the Y31 marker
allele 1 of the VNTR or (+2018) marker of IL-1RN

The 44112332 haplotype comprises the following genotype:

allele 4 of the 222/223 marker of IL-1A
allele 4 of the gz5/gz6 marker of IL-1A
allele 1 of the −889 marker of IL-1A
allele 1 of the +3954 marker of IL-1B
allele 2 of the −511 marker of IL-1B
allele 3 of the gaat.p33330 marker
allele 3 of the Y31 marker
allele 2 of the VNTR marker of IL-1RN

Similarly, three other polymorphisms in an IL-1RN alternative exon (Exon 1ic, which produces an intracellular form of the gene product) are also in linkage disequilibrium with allele 2 of IL-1RN (VNTR) (Clay et al. (1996) Hum Genet 97: 723-26). These include: the IL-1RN exon 1ic (1812) polymorphism (GenBank:X77090 at 1812); the IL-1RN exon 1ic (1868) polymorphism (GenBank:X77090 at 1868); and the IL-1RN exon 1ic (1887) polymorphism (GenBank:X77090 at 1887). Furthermore yet another polymorphism in the promoter for the alternatively spliced intracellular form of the gene, the Pic (1731) polymorphism (GenBank:X77090 at 1731), is also in linkage disequilibrium with allele 2 of the IL-1RN (VNTR) polymorphic locus (Clay et al. (1996) Hum Genet 97: 723-26). The corresponding sequence alterations for each of these IL-1RN polymorphic loci is shown below.

| | Exon 2 | Exon lic-1 | Exon lic-2 | Exon lic-3 | |
|---|---|---|---|---|---|
| | (+2018 of IL- | (1812 of GB: | (1868 of GB: | (1887 of | Pic (1731 of |
| Allele # | 1RN) | X77090) | X77090 | GB:X77090) | GB: X77090) |
| 1 | T | G | A | G | G |
| 2 | C | A | G | C | A |

For each of these polymorphic loci, the allele 1 sequence variant has been determined to be in linkage disequilibrium with allele 1 of the IL-1RN (VNTR) locus (Clay et al. (1996) Hum Genet 97: 723-26).

Linkage disequilibrium between two polymorphic markers or between one polymorphic marker and a disease-causing mutation is a meta-stable state. Absent selective pressure or the sporadic linked reoccurrence of the underlying mutational events, the polymorphisms will eventually become disassociated by chromosomal recombination events and will thereby reach linkage equilibrium through the course of human evolution. Thus, the likelihood of finding a polymorphic allele in linkage disequilibrium with a disease or condition may increase with changes in at least two factors: decreasing physical distance between the polymorphic marker and the disease-causing mutation, and decreasing number of meiotic generations available for the dissociation of the linked pair. Consideration of the latter factor suggests that, the more closely related two individuals are, the more likely they will share a common parental chromosome or chromosomal region containing the linked polymorphisms and the less likely that this linked pair will have become unlinked through meiotic cross-over events occurring each generation. As a result, the more closely related two individuals are, the more likely it is that widely spaced polymorphisms may be co-inherited. Thus, for individuals related by common race, ethnicity or family, the reliability of ever more distantly spaced polymorphic loci can be relied upon as an indicator of inheritance of a linked disease-causing mutation.

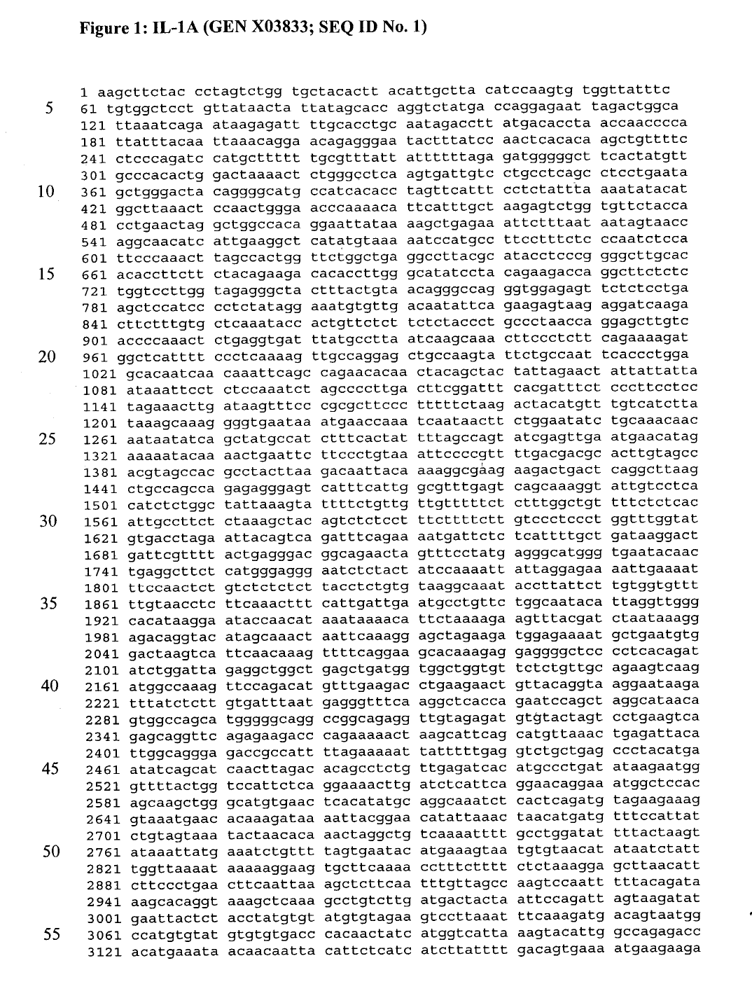

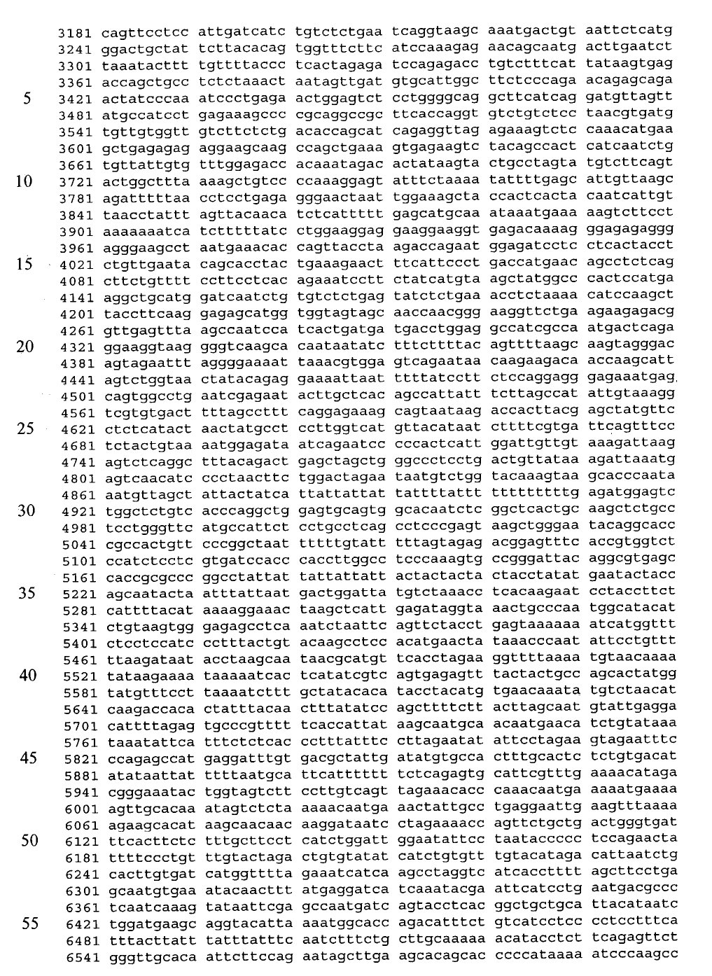

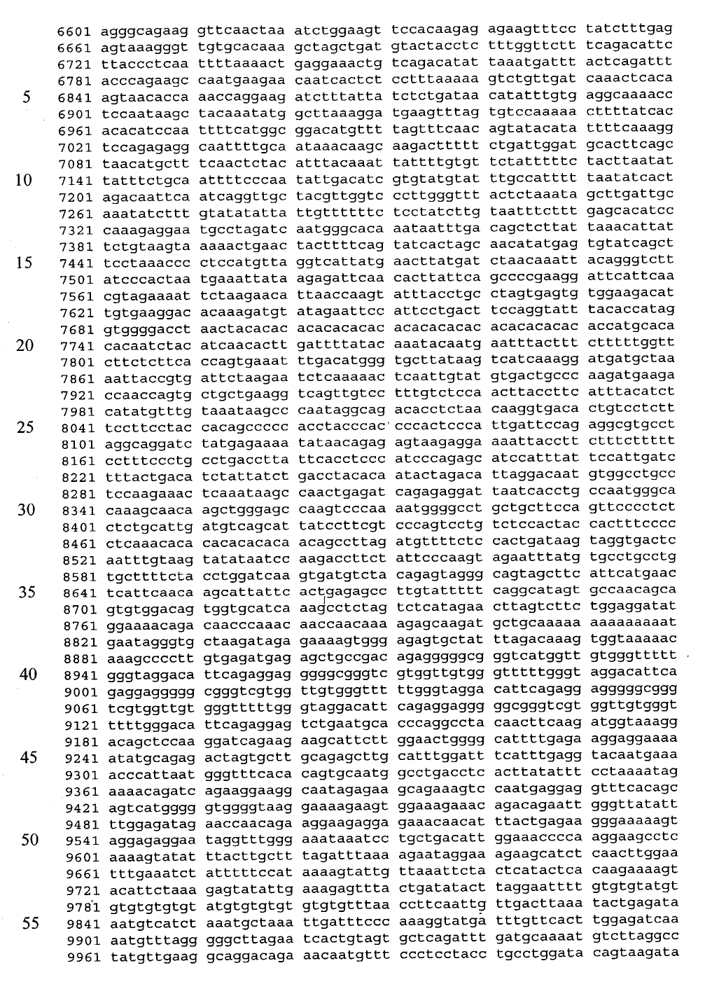

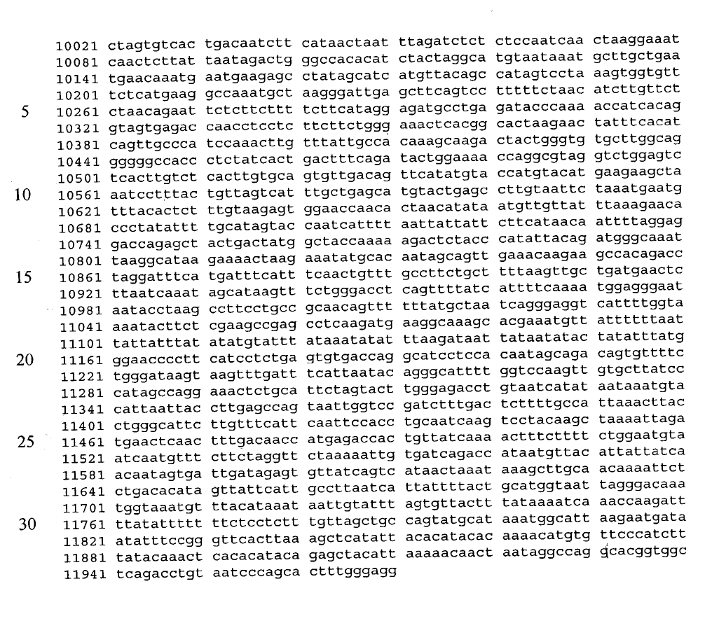

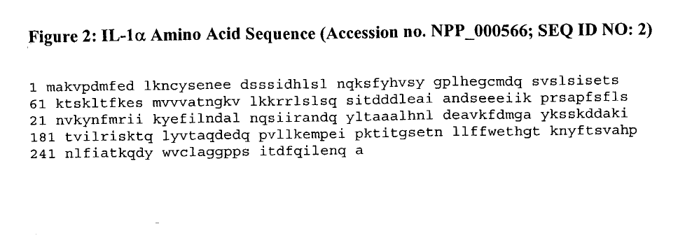

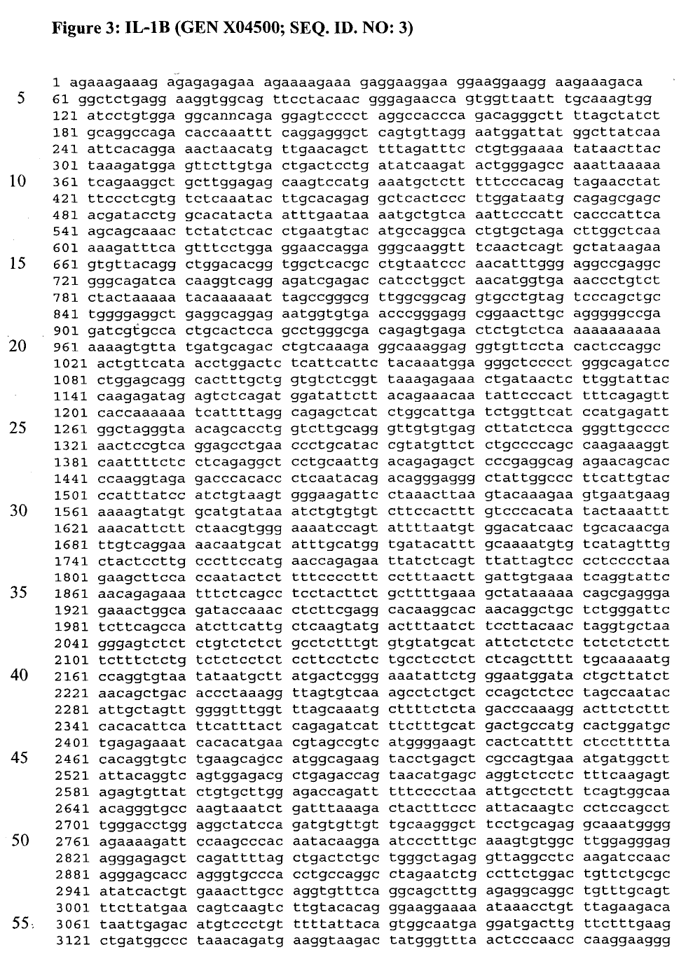

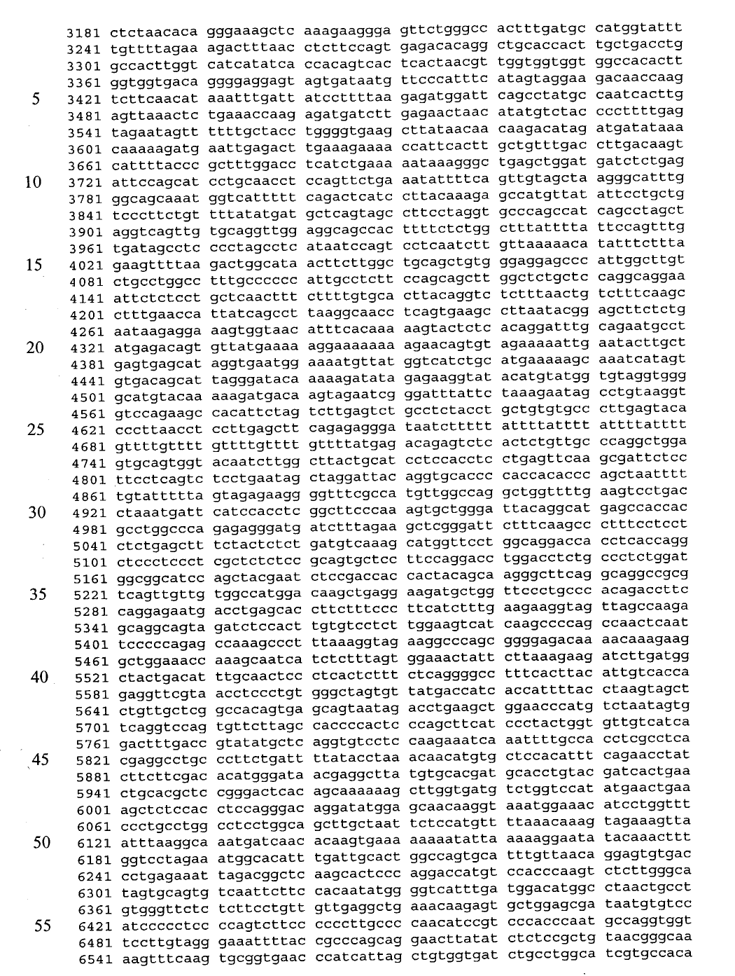

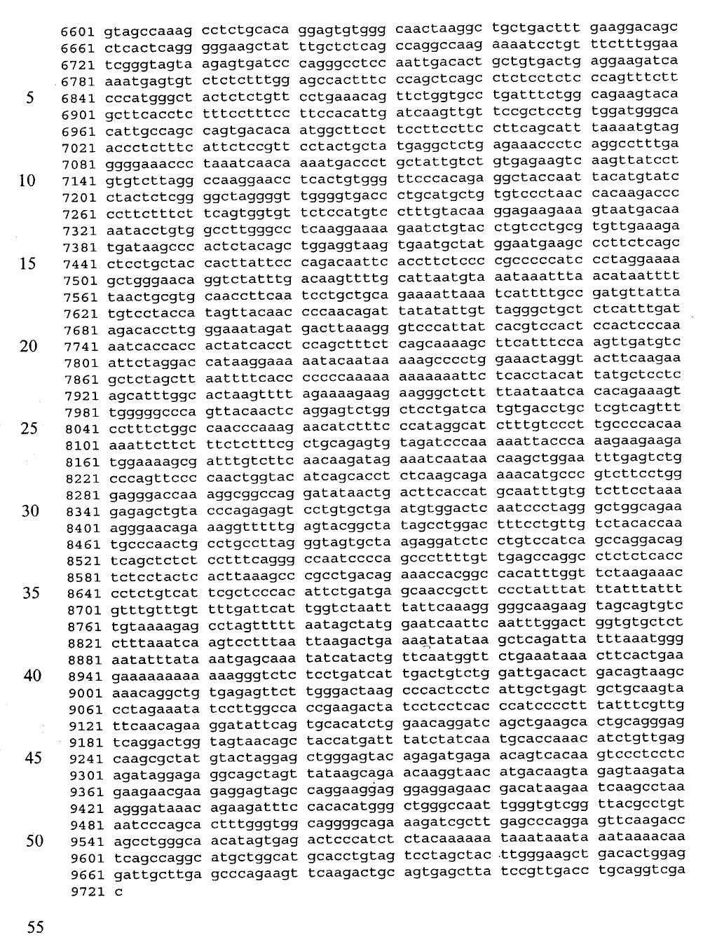

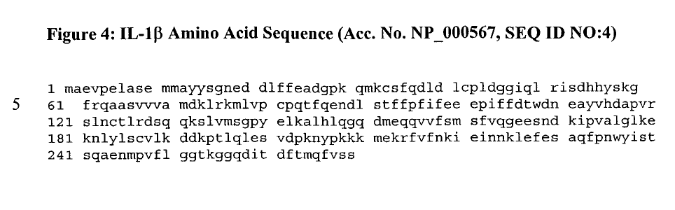

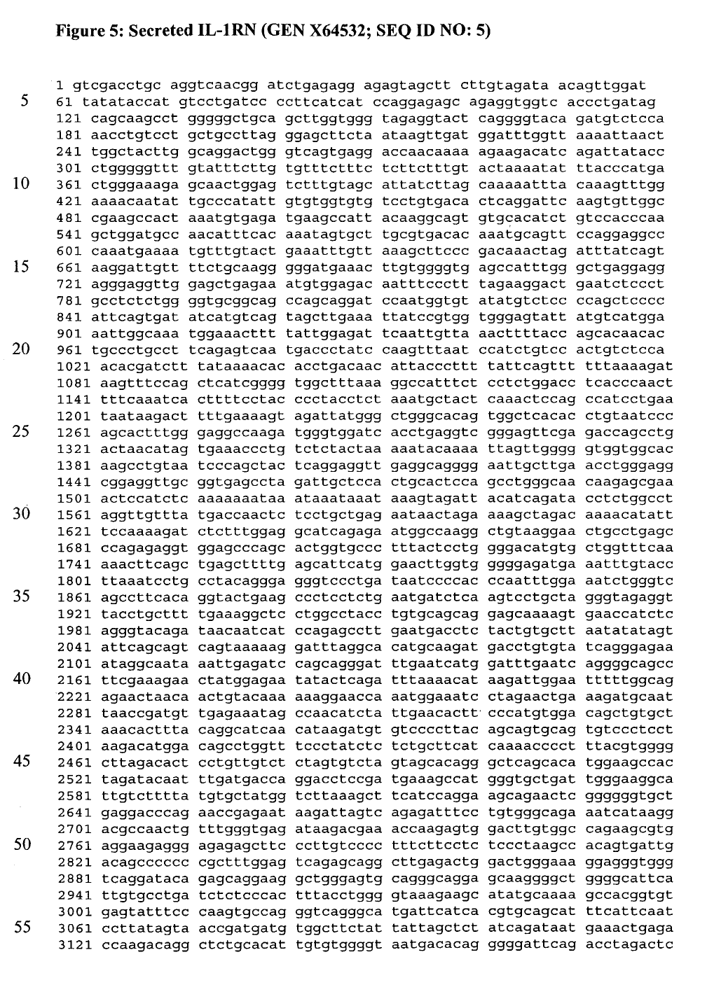

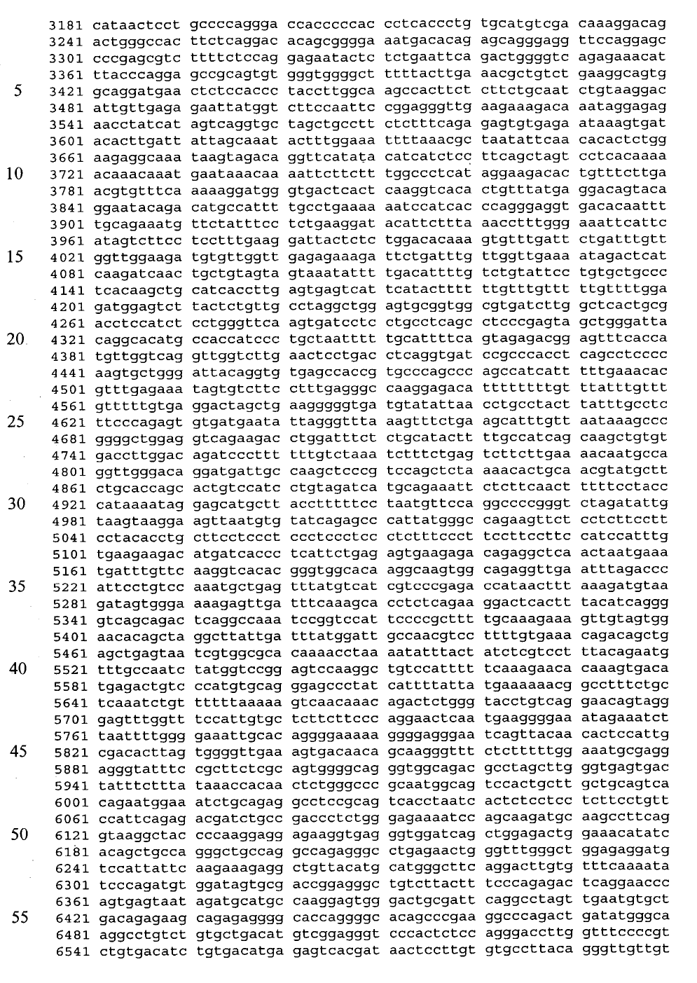

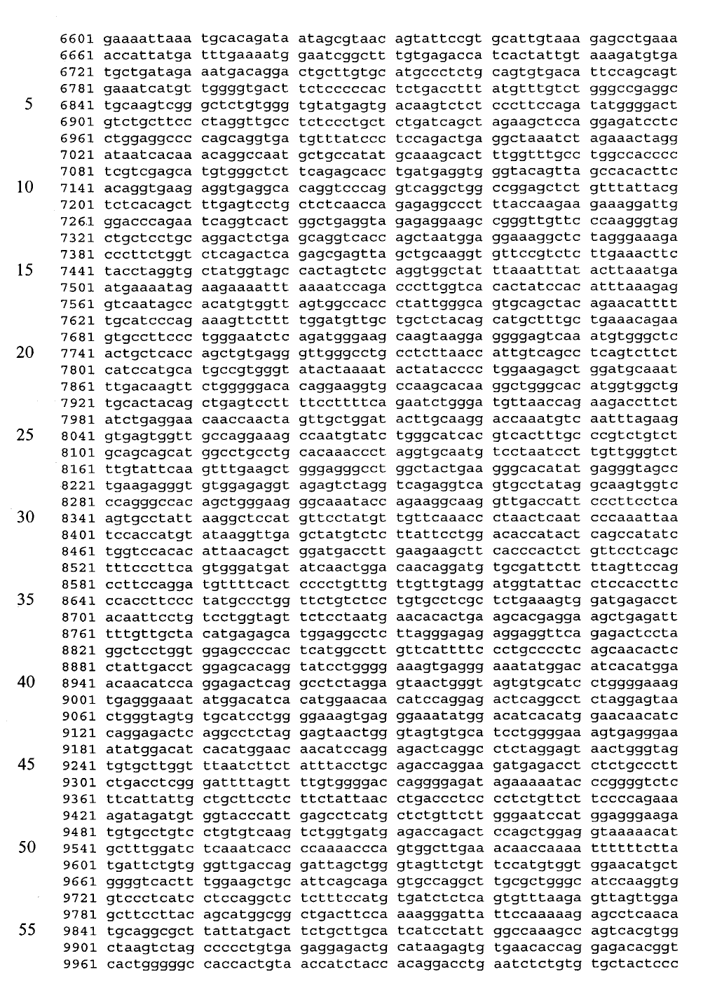

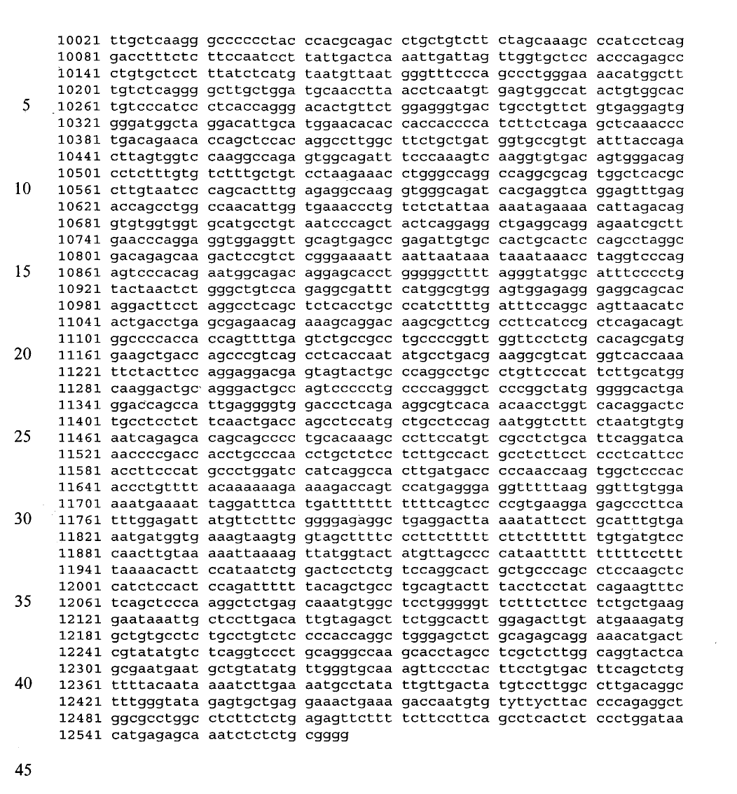

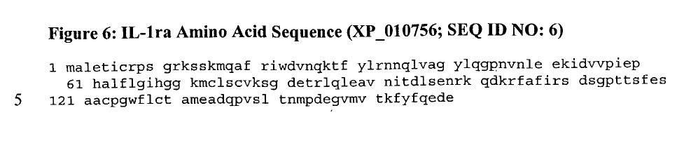

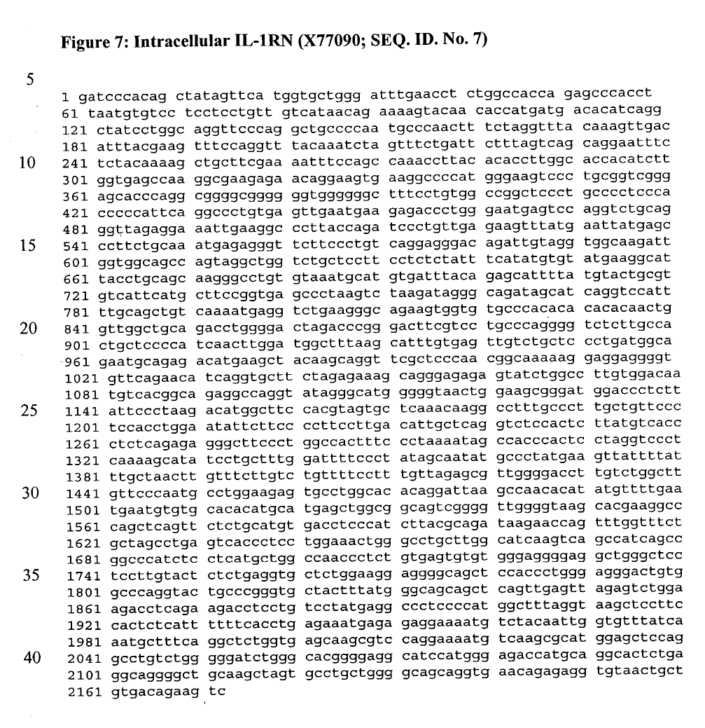

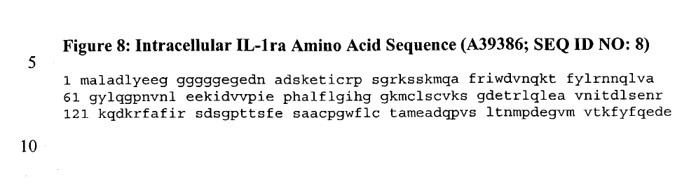

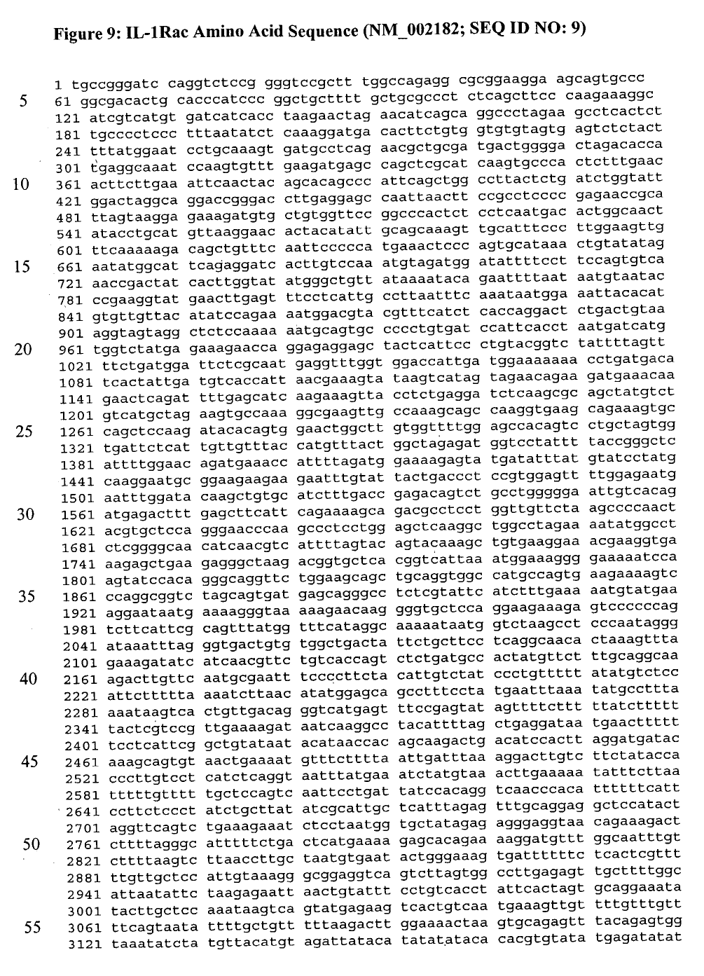

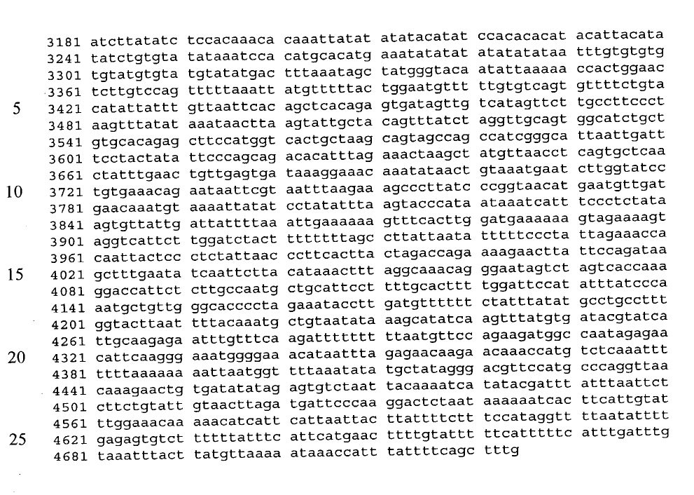

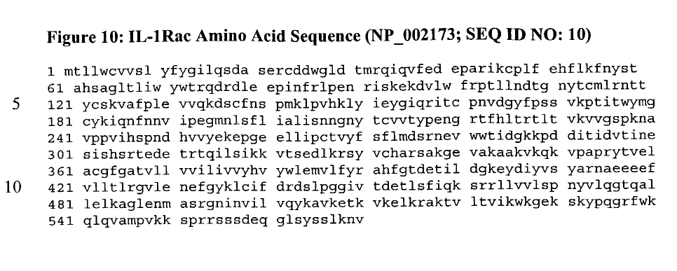

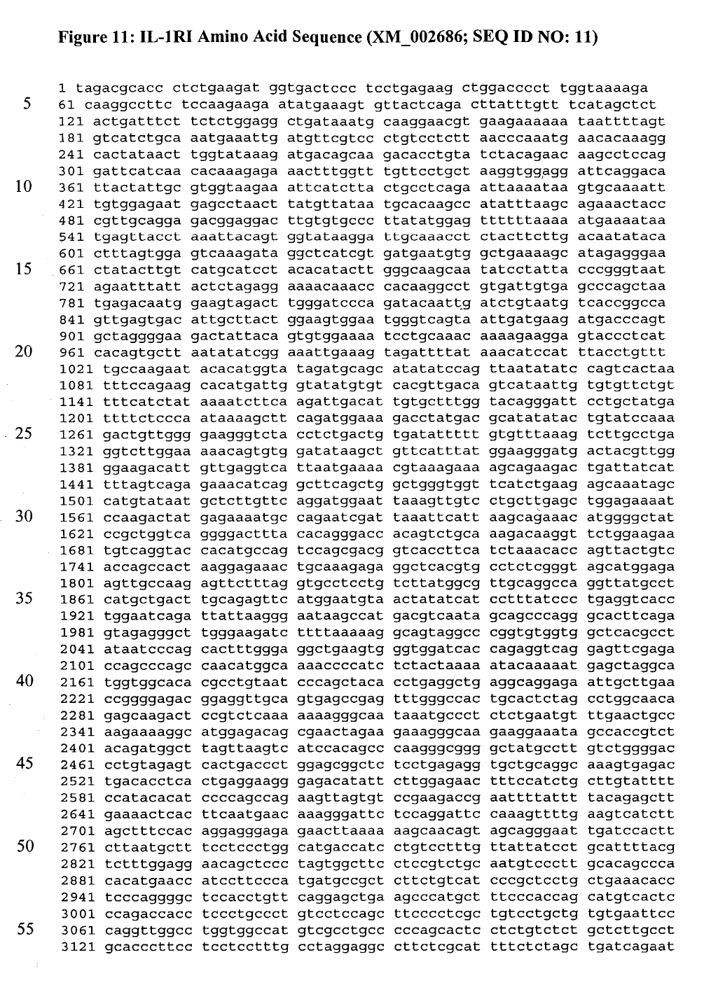

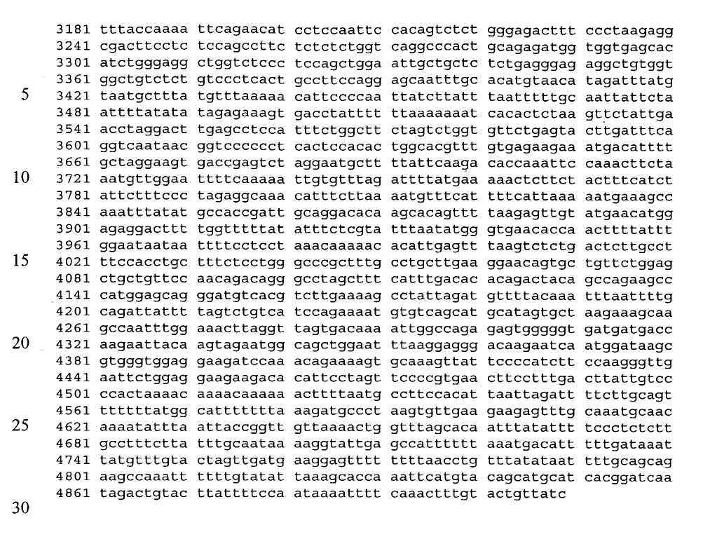

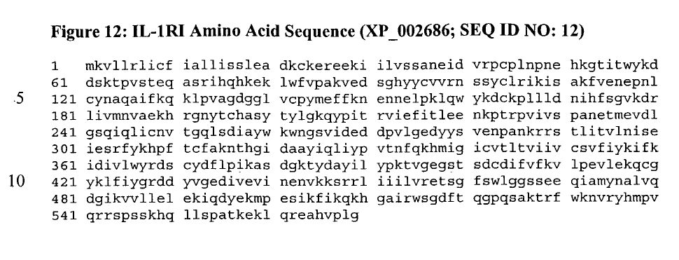

Appropriate probes may be designed to hybridize to a specific gene of the IL-1 locus, such as IL-1A, IL-1B or IL-1RN or a related gene. These genomic DNA sequences are shown in FIGS. 1, 3 and 5, respectively, and further correspond to SEQ ID Nos. 1, 3 and 5, respectively. Alternatively, these probes may incorporate other regions of the relevant genomic locus, including intergenic sequences. Indeed the IL-1 region of human chromosome 2 spans some 400,000 base pairs and, assuming an average of one single nucleotide polymorphism every 1,000 base pairs, includes some 400 SNPs loci alone. Yet other polymorphisms available for use with the immediate invention are obtainable from various public sources. For example, the human genome database collects intragenic SNPs, is searchable by sequence and currently contains approximately 2,700 entries (http://hgbase.interactiva.de). Also available is a human polymorphism database maintained by the Massachusetts Institute of Technology (MIT SNP database (http://www.genome.wi.mit.edu/SNP/human/index.html)). A SNP database is also available through Celera Inc. From such sources SNPs as well as other human polymorphisms may be found.

Accordingly, the nucleotide segments of the invention may be used for their ability to selectively form duplex molecules with complementary stretches of human chromosome 2q12-13 or cDNAs from that region or to provide primers for amplification of DNA or cDNA from this region. The design of appropriate probes for this purpose requires consideration of a number of factors. For example, fragments having a length of between 10, 15, or 18 nucleotides to about 20, or to about 30 nucleotides, will find particular utility. Longer sequences, e.g., 40, 50, 80, 90, 100, even up to full length, are even more preferred for certain embodiments. Lengths of oligonucleotides of at least about 18 to 20 nucleotides are well accepted by those of skill in the art as sufficient to allow sufficiently specific hybridization so as to be useful as a molecular probe. Furthermore, depending on the application envisioned, one will desire to employ varying conditions of hybridization to achieve varying degrees of selectivity of probe towards target sequence. For applications requiring high selectivity, one will typically desire to employ relatively stringent conditions to form the hybrids. For example, relatively low salt and/or high temperature conditions, such as provided by 0.02 M-0.15M NaCl at temperatures of about 50 C. to about 70 C. Such selective conditions may tolerate little, if any, mismatch between the probe and the template or target strand.

Targeted Therapeutics

The ability to rapidly determine the level of a biological activity and/or the genotype of patients promises to fundamentally change the testing, development and use of therapeutic or disease-preventative substances. Currently, the effectiveness of a substance for treating or preventing a disease is assessed by testing it on a pool of patients. While many variables in the patient pool are controlled for, the effects of genetic variability are not typically tested. Consequently, a drug may be found to be statistically ineffective when examined in a genetically diverse pool of patients and yet be highly effective for a select group of patients with particular genetic characteristics. Unless patients are separated by genotype, many drugs with great promise for selected populations are likely to be rejected as useless for the population as a whole. The ability to determine a biological activity, such as, for example, activation state of the IL-1R1 receptor, provides for further refinement in identifying those patients most likely to benefit from a particular therapeutic.

The ability to target populations expected to show the highest clinical benefit, based on a biological activity and or gene profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and 4) more optimal drug administration.

A targeted therapeutic is a modulator of a biological activity measured using an interactive sensor pair. In preferred embodiments the biological activity is receptor signaling. In an exemplary embodiment a targeted therapeutic modulates IL-1 production or signaling. Exemplary inhibitors of IL-1 activity include compositions comprising IL-1ra protein, or active portions thereof (e.g. Anakinra, produced by Amgen Inc.), monoclonal antibodies targeting IL-1α, β or receptor, antisense nucleic acids, etc. In general, preferred therapeutics include nucleic acids, proteins or small molecules.

Transgenic Animals

Transgenic animals can be made for example, to assist in screening for targeted therapeutics. Transgenic animals of the invention can include non-human animals containing nucleic acids encoding an interactive sensor pair, the control of an appropriate promoter or under the control of a heterologous promoter. To compare the effects of different alleles, transgenic animals may be generated with a variety of alleles and differences in phenotype and interactive sensor pair output signal can be identified. Methods for obtaining transgenic non-human animals are well known in the art. In preferred embodiments, the expression of transgenes are restricted to specific subsets of cells, tissues or developmental stages utilizing, for example, cis-acting sequences that control expression in the desired pattern. Toward this end, tissue-specific regulatory sequences and conditional regulatory sequences can be used to control transgene expression in certain spatial patterns. Moreover, temporal patterns of expression can be provided by, for example, conditional recombination systems or prokaryotic transcriptional regulatory sequences.

The transgenic animals of the present invention all include within a plurality of their cells a transgene of the present invention, which transgene alters the phenotype of the “host cell”. In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation of expression of a transgene can be regulated via control of recombinase expression.

Use of the cre/loxP recombinase system to regulate expression of a causative mutation transgene requires the construction of a transgenic animal containing transgenes encoding both the Cre recombinase and the subject protein. Animals containing both the Cre recombinase and a transgene can be provided through the construction of “double” transgenic animals. A convenient method for providing such animals is to mate two transgenic animals each containing a transgene.

Similar conditional transgenes can be provided using prokaryotic promoter sequences which require prokaryotic proteins to be simultaneous expressed in order to facilitate expression of the transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the transactivating protein, e.g. a recombinase or a prokaryotic protein, is delivered to the tissue and caused to be expressed, such as in a cell-type specific manner. By this method, the transgene could remain silent into adulthood until “turned on” by the introduction of the transactivator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals which have one or more genes partially or completely suppressed).

In one embodiment, the transgene construct is introduced into a single stage embryo. The zygote is the best target for microinjection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods. The transgenic animals produced in accordance with the present invention will include exogenous genetic material. Further, in such embodiments the sequence will be attached to a transcriptional control element, e.g., a promoter, which preferably allows the expression of the transgene product in a specific type of cell.

Retroviral infection can also be used to introduce the transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse, Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

Effective Dose

Toxicity and therapeutic efficacy of such compounds can be determined by standard pharmaceutical procedures in cell cultures or experimental animals, e.g., for determining The L_{D}50 (The Dose Lethal To 50% Of The Population) And The E_{D}50 (the dose therapeutically effective in 50% of the population). The dose ratio between toxic and therapeutic effects is the therapeutic index and it can be expressed as the ratio L_{D}50/E_{D}50. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the E_{D}50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulation and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

In clinical settings, a gene delivery system for the a therapeutic gene can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al. (1994) PNAS 91: 3054-3057). An targeted therapeutic gene can be delivered in a gene therapy construct by electroporation using techniques described, for example, by Dev et al. ((1994) Cancer Treat Rev 20:105-115).

The pharmaceutical preparation of the gene therapy construct or compound of the invention can consist essentially of the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Apparatus and Clinical Management

In certain aspects, the invention provides apparatus for measuring the effects of a sample on the output signal of one or more sensor pairs. In some embodiments, an apparatus comprises a sample chamber for receiving a sample, a detection reagent comprising an interactive sensor pair, a mechanism for contacting the detection reagent with said sample, and a sensor capable of measuring the output signal of the sensor pair.

A sample receptacle may be designed to receive the sample material directly or may be designed to hold, receive or couple with a separate sample container. For example, a sample may be placed in a container such as, but not limited to, a flask, cup, conical tube, beaker, cuvette, syringe or the like external to the apparatus, following which the container is placed into the sample chamber of the apparatus. Sample receptacles of the invention may be disposable or reusable. A sample receptacle or separate container may include tubing through which the sample passes. An apparatus may contain a plurality of receptacles into which different samples (or multiple aliquots of the same sample) are placed.

A detection reagent is a composition comprising an interactive sensor pair that may be brought into contact with a sample. The interactive sensor pair may be free in solution, or one or both members of the interactive sensor pair, for example, may be adhered to a substrate, incorporated into a material such as a gel, lipid bilayer or micelle, or expressed in a cell. The detection reagent may include a solid or semi-solid substrate such as beads, plates, fibers, sheets, gels (eg. polyacrylamide, agarose) or any other substrate that permits adherence or incorporation of a member of the interactive sensor pair. The detection reagent may be a solution of soluble and insoluble components. The reagent may, for example, include cells expressing the interactive sensor pair. The cells may be adhered to a substrate or suspended in solution. The reagent may include hydrophobic membranes with a member of the interactive sensor pair inserted therein. Because the output signal depends upon interaction between the members of the interactive sensor pair, it is preferable that a detection reagent not prevent this interaction. A detection reagent may comprise more than one sensor pair to allow simultaneous monitoring of multiple signaling events. Preferably each interactive sensor pair will have a distinct output signal, although it is understood that in certain circumstances it would be desirable to have multiple sensor pairs with the same output signal. Detection reagent sufficient for one or more assays may be stored in the apparatus or provided separately for each assay.

Many mechanisms may be used to bring the detection reagent into contact with the sample, and the mechanism will depend upon the type of sample and the type of detection reagent. For example, the reagent may be a suspension of cells that can be injected into each sample chamber at the appropriate time. The sample chamber may come pre-equipped with detection reagent such that introduction of the sample into the chamber immediately places the sample into contact with the detection reagent. In yet another embodiment, sample may be directed to flow over a surface or gel with the adherent interactive sensor pair.

Sensors to be used depend upon the output signal to be detected. Sensors may include fluorimeters or fluorescence microscope setups with appropriate excitation and detection spectra. In view of this specification, one of skill in the art would be capable of selecting an appropriate sensor.

In other aspects, the invention provides apparatus that provide a therapy in a dosage that is determined by the effects of a patient sample on one or more interactive sensor pairs. In certain embodiments, the apparatus would resemble that described above with the addition of a therapeutic agent and a means for administering the therapeutic to a patient. The apparatus includes a control connection between the sensor and a dosing element for administering the therapeutic so that the dosing is regulated in response to the signal output from the detection reagent. Dosing may be controlled in terms of amount and/or timing of each dose. Dosing may be altered in response to any change, whether up or down, in the output signal of a sensor pair.

For example, many of the fatal complications that result from sepsis are stimulated by an excessive level of IL-1 and TNFα signaling. An exemplary apparatus may include a line for repeated blood sampling from a patient with severe sepsis. The blood samples would be tested against interactive sensor pairs to assess the level of IL-1 and TNFα activating signals in the blood. The apparatus may have one or more reservoirs with therapeutics for controlling the effects of IL-1 and TNFα signaling, and the amount of these therapeutics administered to the patient could be continuously adjusted to respond the level of IL-1 and TNFα activating signals in the blood.

### EXAMPLES

The invention now being generally described, it will be more readily understood by reference to the following examples, which are included merely for purposes of illustration of certain aspects and embodiments of the present invention, and are not intended to limit the invention.

### Example 1: IL-1RI/IL-1Rac Interactive Sensor Pair

Introduction

A 430-kb region of human chromosome 2q1.2 contains three known IL-1 genes (A, B, RN). Several multiallelic and bi-allelic (SNP) variations have been characterized in and around these genes and linkage disequilibrium has been measured as moderate to high across the region (−0.8). Common haplotypes in Caucasian populations have been identified. A large weight of evidence points to a key role for this gene system in the regulation of the inflammatory response.

Certain haplotypes of the IL-1 gene cluster have been associated with inflammatory disease phenotypes in clinical populations, and at least one family-based study has detected significant linkage to this region in rheumatoid arthritis. Many of the association studies have been performed in large populations and have been replicated in independent populations. Most association studies have been performed using representative markers of definable haplotypes and odds ratios in the range 2-10 have been obtained in important public health conditions such as periodontitis, post-menopausal osteoporosis, early-onset ischaemic heart disease, diabetic complications, asthma, inflammatory bowel disease, inflammatory dermatoses, Alzheimer's Disease and certain cancers.

Commonly, associations have been analyzed by carriage of at least one copy of an allele because homozygosity of rarer alleles is relatively uncommon and study populations need to be very large to provide power to analyze the odds ratio of association with homozygous rarer alleles of a SNP. However, when such analysis has been possible, homozygosity for the associated allele has usually demonstrated a higher odds ratio for disease association than heterozygosity, suggesting a gene dose effect.

The odds ratios that have been observed, even by single copy carriage analysis, are sufficiently large to provide clinical utility in patient management. For example the observations provide a basis for re-classification of disease subsets in terms of prognosis, optimal treatment choices and schedules, and the design of preventive strategies in healthy ‘at risk’ individuals. The genetic leads also provide strategies for target identification for new drug design as well as helping to define the licensed indications for new and existing therapeutics.

Evidence is also accruing that disease risk associated with IL-1 genotype interacts with conventional risk factors such as lipid profiles in atheromatous disease, smoking in periodontal disease and bacterial serotype in meningococcal disease.

A more complete risk matrix will lead to more accurate clinical prognosis in a wide range of inflammatory diseases (where dysregulated inflammation is thought to be a primary or important etiological factor) and in diseases where the inflammatory response contributes to survivability and pathology (such as infectious disease and cancer).

The precise mechanisms underlying gene associations in clinical populations remain incompletely understood. However, there is a strong biological rationale for believing that the IL-1 system is a key regulator of the innate inflammatory response and there is evidence that certain sequence variants of IL-1 genes are themselves associated with altered levels of gene product.

The difficulty in analyzing the functional contribution of allelic variation in the IL-1 system to the genetic epidemiological observations in disease populations arises from several complicating factors:

(1). There may be other genes in linkage disequilibrium with the IL-1 cluster that contribute to the genetic epidemiology observations. Such genes might include both IL-1-related and IL-1-unrelated genes.

(2) Epistasis: The biological activities of the protein products of all three known IL-1 genes are thought to be mediated by a single class of cellular receptor, type 1 IL-1 receptor (IL-1R1). The IL-1A and IL-1B genes encode proteins that act as IL-1R1 agonists, and IL-1RN encodes an antagonistic protein for this receptor. The cellular response mediated by IL-1R1 is, therefore, determined by a ratio of agonistic to antagonistic ligands. This implies that the biological relevance of any given IL-1 haplotype is a resultant property of all functional polymorphisms in linkage disequilibrium that form that haplotype.

(3) There are other classes of IL-1 receptor on the cell surface membrane and in extracellular fluid that do not signal but bind IL-1 proteins and therefore act as inhibitors of IL-1 activity (eg type 2 IL-1 receptor). The contribution of soluble IL-1 binding proteins to regulation of IL-1 responses in vivo is poorly understood.

(4) The binding affinity of IL-1 proteins for the type 1 IL-1 receptor is altered by proteolytic processing, most notably in the case of IL-1β where the initial translation product (31 kd) has low receptor affinity and is biologically inactive, but the mature 17 kd protein has high receptor binding affinity and biological activity. The proteolytic processing of IL-1β is performed by Interleukin-l converting enzyme (ICE).

(5) There may be alternatively spliced forms of the gene products with altered biological activity.

(6) There may be post-translational processes that produce IL-1 species with different biological activities.

(7) Transcriptional, translational and post-translational processes may be tissue specific, ontogeny-related, or specific to the signaling pathway to IL-1 gene regulation (ie stimulus -specific).

For these reasons and others, the analysis of individual polymorphisms in IL-1 genes can unrewarding and complex in terms of predicting the overall biological significance of a particular IL-1 gene cluster haplotype.

IL-1R1 signals to the nucleus along a kinase pathway that is not exclusive, in other words the signaling pathways from other receptors on the cell surface (eg TNF, PDGF, IL-6 receptors) feed into a common pathway with IL-1 signaling. Thus, to exclude interference with other cellular products inevitably present in the supernatants of the tested cellular population, a quantitative and measurable event that is specific for IL-1R1 signaling must be identified. Such an event is most likely to be very proximal to the IL-1R1, occurring early in the signaling cascade from that receptor.

A test system that would respond quantitatively and reproducibly to all functional polymorphisms contributing to the biological output of the IL-1 gene system would offer major advantages in understanding the relationship between genotype and functional phenotype. This, in turn, would begin to provide a biological rationale to explain the disease associations with various IL-1 genotypes in different disease populations.

Such a test system may be constructed around the response of the type 1 IL-1 receptor to the products of cells of known IL-1 haplotype. A standardized and immortal cell line bearing functional IL-1R1 receptors could detect soluble factors by supernatant transfer and cell-associated factors by cell-to-cell.

Proposed Novel in vitro Test System

One of the earliest molecular interactions to occur following IL-1 agonist binding to the IL-1R1 is the recruitment of IL-1R accessory protein (IL-1Rac) to form a complex with the C terminal (cytosolic) domain of IL-1R1. According to our current understanding, this molecular association is an essential early step in signaling from IL-1R1 to the cell nucleus and may be regarded as a surrogate measure of IL-1R1 signaling and therefore the biological activity of the receptor. Importantly, binding of IL-1 receptor antagonist to the extracellular domain of IL-1R1 does not cause recruitment of IL-1Rac to the IL-1R1 cytosolic domain. A method to visualize the degree of IL-1R1 complexing with IL-1Rac in a target cell therefore could provide a measure of IL-1 ligand binding to the extracellular domain of IL-1R1 and would integrate the activity of the IL-1 gene system in test cell populations giving a resultant biological significance.

Visualizing IL-1R1 Association with IL-1Rac

We have constructed nucleic acid constructs encoding fusion proteins comprising IL-1R1 and jellyfish green fluorescent protein (GFP). The DNA constructs can be expressed in stably-transfected cell lines and emit measurable fluorescence at the single cell level detectable by con-focal microscopy. IL-1Rac can also be engineered as a fusion protein with GFP and can be co-transfected into the same cells with IL1R1-GFP.

The detection of complex formation between these two fusion proteins requires optimally a qualitative change in the fluorescence emission, specific to complex formation. Such an event could be engineered by using two different GFP mutant genes with different spectral properties. Such mutants exist and provide an excellent approach to achieving this aim based on fluorescence resonant energy transfer (FRET). In the system we envisage, fusion proteins comprising cyan-GFP (blue) with IL-1R1 and yellow-GFP with IL-1Rac would be used. Cyan is the donor partner and yellow the acceptor partner in the FRET pair. When the molecules are brought together (as would happen when IL-1R1 complexes intracellularly with IL-1Rac following extracellular ligand binding), donor-enhanced acceptor emission and acceptor quenched donor emission occurs resulting in progressive darkening of the donor partner and brightening of the acceptor partner. Increasing binding of IL-1 agonists to the IL-1R1 would result in a progressive increase of yellow emission from these cells as complexes form between IL-1R1 and IL-1Rac. This yellow emission is quantifiable (initially by confocal microscopy) at the single cell or whole field level. Importantly, for assay development, the changing emission pattern would be detectable in a period of around 30 minutes.

### Example 2: Early Detection of Infectious Exposure

Rapid early assessment of exposure to infectious agents is useful so that a course of therapy can be initiated in an expeditious manner. With exposure to most biological agents, treatment is particularly effective at an early stage of the infection. With infectious challenge to the body, among the first host genes activated are interleukin-1 (IL-1) and tumor necrosis factor alpha (TNFa). These cytokines activate multiple cascades of gene activation that serve to rapidly amplify both the local and systemic host-response mechanisms. IL-1 and TNFa are believed to be ancient molecules in the evolutionary development of biological systems, and therefore represent an early response mechanism that is non-specific. Because of their early response and non-specific activation, these specific cytokines appear to be ideal biomarkers for screening for an infectious or toxic challenge to the host. In addition plasma concentrations of IL-1b and TNFa correlate well with the severity of infection and with septic shock.

The net IL-1 biologic activity is an integration of interactions among multiple agonists, antagonists, and receptors. The proinflammatory effects of IL-1 are mediated by IL-1α that remains mainly cell-associated and by IL-1β that is mostly released. These effects are controlled by the natural antagonist, IL-1Ra, a protein that binds to IL-1 receptors but lacks agonist activity. IL-1 activity is also modulated by the density of cell-bound IL-1 receptors (IL-1R) and the presence of IL-1 soluble receptors (sIL-1R) that are generated by cleavage of the extracellular domain of IL-1R.

In addition, two types of IL-1 receptor exist- the IL-1R type I is predominately expressed on the surface of T cells and fibroblasts, and the IL-1R type II is expressed on the surface of B cells and monocytes. The two receptors demonstrate differential affinities for the ligands. For example, the type I receptor affinity preference is IL-1Ra>IL-1α>IL-1β, whereas, the type II receptor affinity preference is IL-1β>IL-1α>IL1Ra. Transmission of IL-1 signals occurs via IL-1RI where as IL-1RII functions as “decoy” receptor.

In physiologic samples the varying relative concentrations of IL-1α, IL-1β, IL-1Ra, and sIL-RII would, therefore, determine the net stimulation of the IL-1 receptor. Upon binding of IL-1α or IL-1β to the IL-1R, a cascade of secondary messengers is activated that ultimately lead to perturbations in the expression of a host of IL-1 target genes. Several of these secondary messenger activation events, such as, increased cAMP levels and activation of MAP kinases, also occur when receptors for TNFa, Toll, secondary cytokines or growth factors are stimulated and therefore are not a good measure of the definite activation of the IL-1 system. IL-1 is a comparatively simple gene system, but decoding the physiology of these six interacting gene products in vivo is beyond the limits of existing technologies.

The early response cytokines, such as IL-1 and TNFa, show large fluctuations in serum concentration over time and therefore measurement of the cytokines levels, by themselves, do not provide a reliable assessment of a response to an infectious challenge and present technical problems. In addition, current technology to measure cytokine concentration in any biological samples requires complex equipment and assays that require multiple washing steps and are not favorable for a rapid measurement in a field situation. Other cytokines, such as IL-6, exhibit less variability than the early response cytokines but have similar complication in terms of the logistics of assay conditions.

Furthermore, immunoassays for cytokines only measure the concentration of the proteins but do not measure their biological activity. On the other hand, existing bioassays, which are biologically more meaningful, may not be specific to the early response cytokines receptor activation. Many of the bioassays measure some components of the downstream signal transduction that is not unique to early response cytokines but occurs when growth factor or secondary (late response) cytokine receptors are stimulated. However, if a key event that is both specific for the IL-1/IL-1R system and is also a high-fidelity surrogate of physiological output of the interaction, is measured then the limitations of existing technologies (immunoassays and bioassays) can be avoided. IL-1 signaling is initiated by the interaction of the type I IL-1 receptor with an accessory protein (AcP). This interaction occurs when the IL-1α or IL-1β binds but not when IL-1Ra binds to the receptor. The contact between IL-1 receptor and the IL-1Ac protein is a key molecular association event that reflects the net stimulation of the IL-1 receptor. Therefore, a goal of the IL-1 bio-integrative assay is to measure the molecular activation of the IL-1 receptor with substantial selectivity, which would be a reflection of the total IL-1 bioactivity in the sample.

Therefore, the biologically significant relative levels of IL-1 agonists and antagonists may be assayed by determining the activation of the IL-1R1-AcP complex. The assay was designed to monitor this protein-protein interaction in a relatively specific and quantifiable manner, employing technology that is adaptable to a rapid assay system for field use. We have identified what we believe to be a key molecular association that integrates the information from molecular interactions between known IL-1 ligands and receptors and is a surrogate of IL-1 biological output. Moreover, it is absolutely specific for the IL-1 system, is quantitative, and measurable,

This assay involves recombinant proteins that are fluorescence tagged and incorporated into a matrix. The system assays the net IL-1 biological activity that is an integration of multiple ligands (IL-1α, IL-1β) and multiple inhibitors (IL-1ra, IL-1 soluble receptors) as they interact with the IL-1 receptor. The technology is adaptable to a solid-state format.

The assay may also be incorporated into systems (i.e. kits). An exemplary system has the following elements:

1. a finger-stick to collect a drop of blood

2. an assay strip with all reactants integrated on it.

This system requires no processing of the sample (although the sample may be processed or fractionated as desired), and the assay strip may be read immediately in a hand-held reader. The output is a quantitative assessment of net IL-1 biological activity, thresholds, based on clinical studies, will identify probability of active infectious challenge

A further exemplary system has a micro-reader that is in-dwelling adjacent to a capillary bed and a transponder that would transmit an alert when a biomarker profile indicated exposure to an infectious threat.

### Example 3: FRET Using IL1-R1-CFP and IL1-ac-YFP

IL-1R1 measurements were demonstrated by employing fluorescence resonance energy transfer (FRET) between labeled molecular species involved in signaling IL-1 receptor activation.

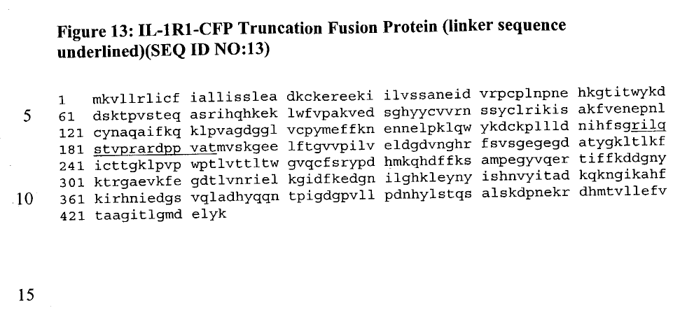

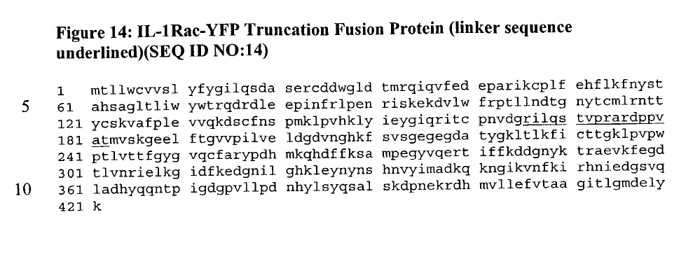

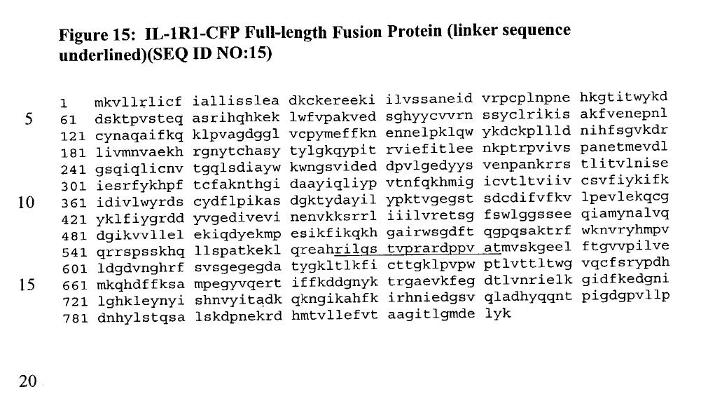

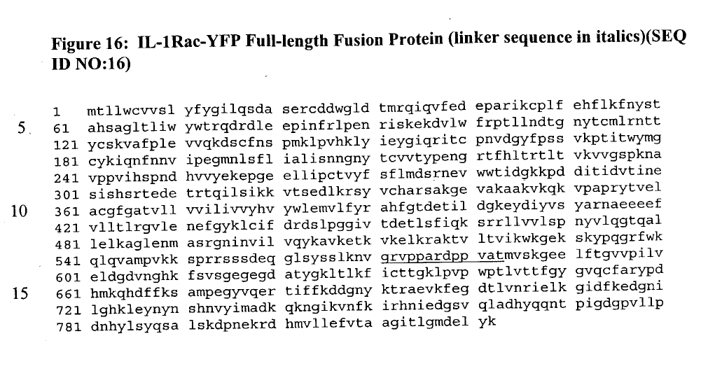

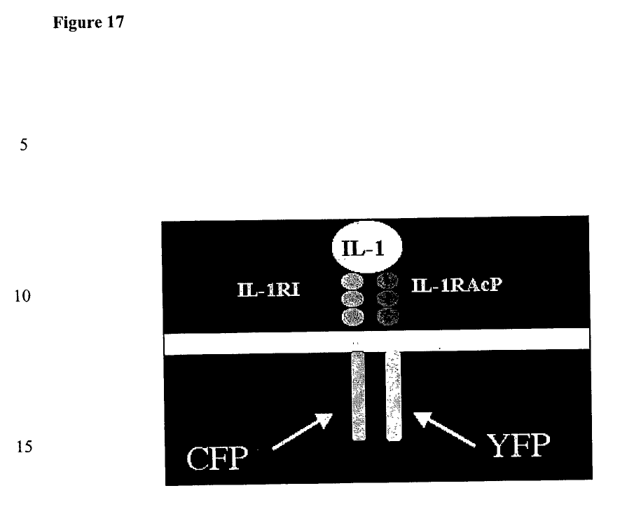

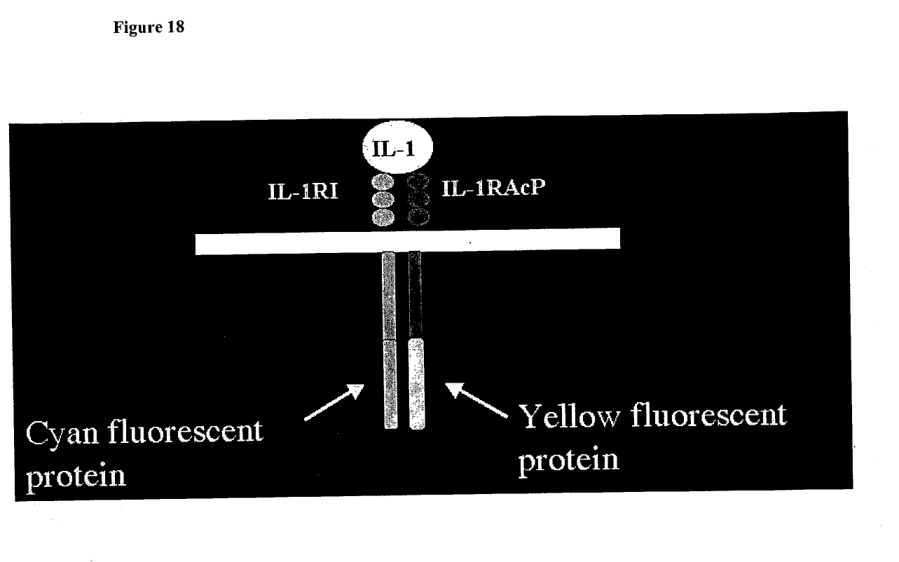

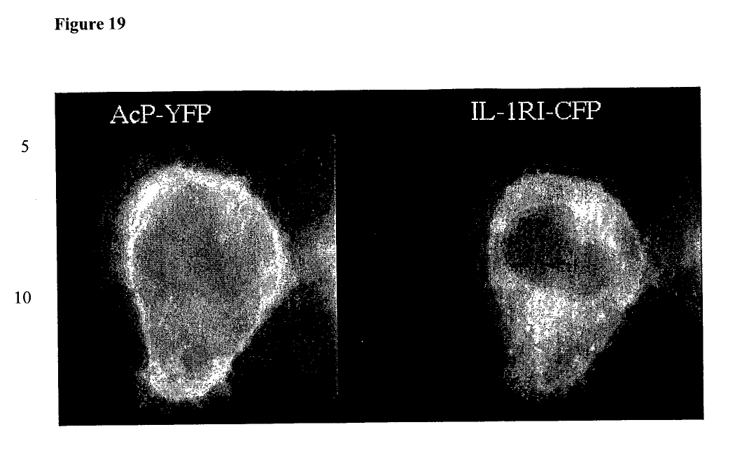

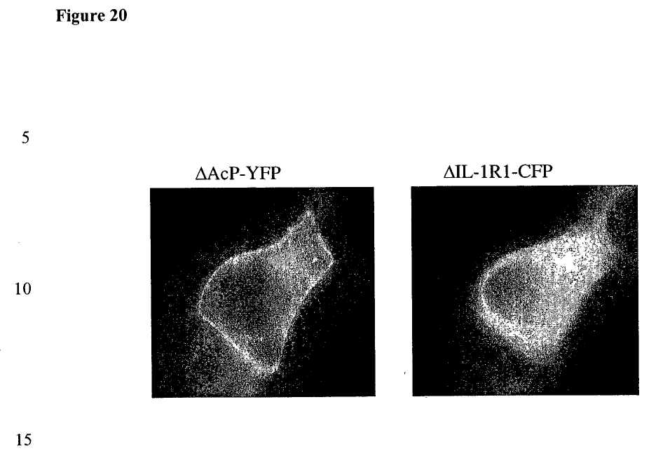

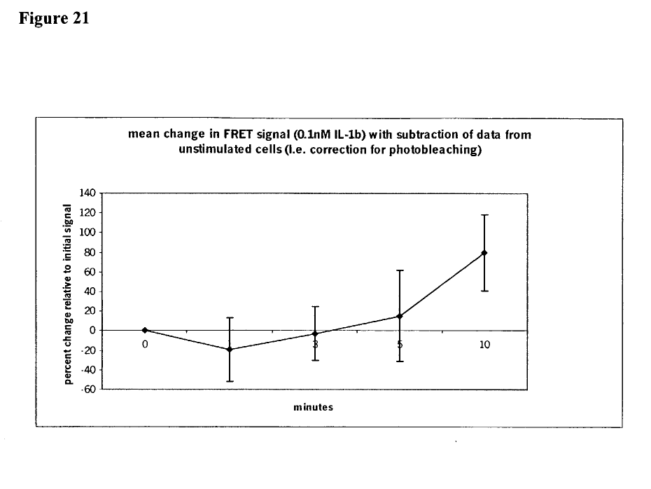

We constructed a nucleic acid translational fusion between a truncated version of the IL-1 receptor (lacking most of the cytoplasmic domain—containing amino acids 1-176 of SEQ ID NO: 12) and a cyan fluorescent protein (CFP), with a linker (RILQSTVPRARDPPVAT) in between (FIG. 13). Similarly, we constructed a nucleic acid translational fusion between a truncated version of the IL-1 receptor accessory protein (amino acids 1-165 of SEQ ID NO:10) was labeled with yellow fluorescent protein (YFP), with a linker (RILQSTVPRARDPPVAT) in between (FIG. 14). In HEK293T cells co-expressing these fluorescent receptor fusions, an IL-1-induced change in the FRET signal was observed. We have obtained HEK293 cells stably transfected with these fusion constructs. Fusions comprising the full-length IL1-R1 and IL1-Rac with CFP and YFP, respectively, were also constructed (linker sequence GRVPPARDPPVAT for IL1-Rac, FIG. 16, RILQSTVPRARDPPVAT for IL1-R1, FIG. 15). Other exemplary IL1-R1 fusion proteins are generated by truncating the IL-1R1 sequence at amino acid 92, 460 or 517.

Experiments with cells transfected with the genes for the fusion proteins have shown that the hybrid proteins retained the desired properties of specific, IL-1-dependent association, and that the association could be detected by FRET, as measured by confocal microscopy.

Transfected cells were treated with varying concentrations of IL-1a, IL-1b and IL-1 Ra alone or in combination over several time points Interactions between the IL-1R and IL-1Rac were measured by FRET.

We have further developed a high throughput assay system using multi-well plate and automated luminometer, where the plate reader has been calibrated to detect FRET signal. In addition, detection may be performed using a FACS system.

Vectors for the expression and purification of milligram quantities of His-tagged receptor fusion proteins are being developed.

### Example 4: Bioluminescence Resonance Energy Transfer

A BRET (Bioluminescence resonance energy transfer) based system uses resonance energy transfer between Renilla Luciferase (Rluc) and GFP. Spectral resolution in the BRET system (105 nm) is about twice that obtained with CFP/YFP FRET, thus increasing sensitivity and dynamic range. The carboxy terminus of the IL-1 receptor and Ac protein are fused to Rluc and GFP respectively. IL-1 induced receptor-Ac protein interaction are detected by measuring BRET.

### Example 5: Luminescence Based System

An exemplary luminescence based system utilizes the well-known bacterial enzyme, beta-galactosidase, the gene product of LacZ. The functional beta-galactosidase enzyme is a tetramer consisting of 4 identical subunits. Several domains from each polypeptide chain are involved in the formation of this tetrameric enzyme. Restoration of the enzymatic activity occurs when the a-mutant combines with a second mutant form of beta-galactosidase to provide the structure necessary to form an intact beta-galactosidase complex. This phenomenon is called intra-cistronic complementation or alpha-complementation. The enzyme activity that results from beta-galactosidase complementation is a direct measurement of the protein-protein interaction. The carboxy terminus of the IL-1 receptor and Ac protein will be fused to the mutants of the beta-galactosidasel enzyme subunits. An IL-1-induced IL-1R-Ac protein interaction will cause beta-galactosidase enzymatic activity. A variety of beta-galactosidase substrates that yield a fluorescent molecule are available (e.g. 3-carboxyumbelliferyl β-D-galactopyranoside, Molecular Probes, Inc., Eugene, Oreg.) and by using such a substrate, a luminescence measurement correlates with IL-1R1 activity.

Various infectious challenges will be performed in animal models. In addition, human clinical models will also be used to assay responses to both bacterial challenges and mechanical trauma.

### Example 6: Cell-free System

An exemplary cell-free system employs a system of FRET-suitable fusion proteins inserted in a membrane layer. Vectors are constructed for the expression and purification of His-tagged receptor fusion proteins. For example, the Gateway system from Invitrogen (Carlsbad, Calif.) provides a number of useful vectors for preparing expression constructs. The pDEST26 vector provides an N-terminal hexahistidine tag for ease of purification, and expression of the fusion gene in mammalian cells is driven by a CMV (cytomegalovirus) promoter. The pT-ReX-DEST31 vector provides for inducible expression of a fusion protein carrying an N-terminal hexahistidine tag. Expression is driven by a CMV promoter but regulated by Tet operator sequences, that mediate repression of transcription by the Tet repressor protein. Expression may be induced with tetracycline. pMT-DEST48 is useful for expression in insect cells (e.g. Drosophila S2 cells). The vector provides an N-terminal hexahistidine tag and expression is driven by the Drosophila metallothionein promoter, which is inducible with copper sulfate or cadmium chloride.

Purified proteins may are reconstituted in a unilamellar bilayer membrane, such as may be generated from a standard phosphatidylcholine membrane mixture, commercially available (e.g. Avanti Polar Lipids, Inc., Alabaster, Ala.).

Alternatively, purified proteins are incorporated into substrates such as nitrocellulose, or liquid crystalline.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The fill scope of the invention should be determined by reference to the claims, along with their full scope of equivalents, and the specification, along with such variations.

1. A detection reagent comprising an interactive sensor pair, the interactive sensor pair comprising a first polypeptide and a second polypeptide, wherein the first polypeptide comprises a multiple-ligand-responsive receptor, and wherein the second polypeptide comprises a polypeptide that binds to the first polypeptide, the binding being affected by binding of a ligand to the first polypeptide, and wherein the first polypeptide or the second polypeptide is stably attached to a reactive module.

2. The detection reagent of claim 1, wherein the first polypeptide is stably attached to a reactive module and the second polypeptide is stably attached to a reactive module.

3. The detection reagent of claim 1, wherein the multiple-ligand-responsive receptor is a transmembrane receptor.

4. The detection reagent of claim 1, wherein the multiple-ligand-responsive receptor is a cytokine receptor.

5. The detection reagent of claim 1, wherein the multiple-ligand-responsive receptor is an IL-1R-like receptor.

6. The detection reagent of claim 1, wherein the multiple-ligand-responsive receptor is IL-1RI or an active portion thereof.

7. The detection reagent of claim 1, wherein the second polypeptide comprises IL-1Rac or an active portion thereof.

8. The detection reagent of claim 2 wherein the first reactive module and the second reactive module are fluorescent molecules capable of exhibiting fluorescence resonant energy transfer.

9. The detection reagent of claim 1, wherein the reactive module comprises a polypeptide selected from the group consisting of: a fluorescent protein and an enzyme.

10. The detection reagent of claim 1, wherein at least one of the first and second polypeptides is associated with a lipid layer.

11. The detection reagent of claim 1, wherein at least one of the first and second polypeptides is associated with a cell.

12. The detection reagent of claim 1, wherein at least one of the first and second polypeptides is associated with a solid or semi-solid substrate.

13. The detection reagent of claim 1, wherein at least one of the first and second polypeptides is a purified polypeptide.

14. A kit comprising a detection reagent of claim 1.

15. The kit of claim 14, further comprising an implement for obtaining a sample from a subject.

16. A cell comprising a first nucleic acid and a second nucleic acid, the first nucleic acid comprising a coding sequence for an IL-1R-like receptor, or an active portion thereof, translationally fused to the coding sequence for a polypeptide reactive module, and the second nucleic acid comprising a coding sequence for an IL-1Rac, or an active portion thereof, translationally fused to the coding sequence for a polypeptide reactive module.

17. The cell of claim 16, wherein the IL-1R-like receptor comprises the amino acid sequence shown in SEQ ID NO:12 and wherein the IL-1Rac comprises the amino acid sequence shown in SEQ ID NO:8 or 10.

18. A method for measuring the ability of a sample to modulate a receptor comprising
placing the sample in the presence of a detection reagent comprising an interactive sensor pair, the interactive sensor pair comprising a first polypeptide and a second polypeptide, wherein the first polypeptide comprises a multiple-ligand-responsive receptor, and wherein the second polypeptide comprises a polypeptide that binds to the first polypeptide, the binding being affected by binding of a ligand to the first polypeptide, and wherein the first polypeptide or the second polypeptide is stably attached to a reactive module; and
measuring the output signal;
wherein a change in output signal indicates that the sample modulates the transmembrane receptor.

19. The method of claim 18, wherein the first polypeptide is stably attached to a reactive module and the second polypeptide is stably attached to a reactive module.

20. The method of claim 18 wherein the sample comprises a sample from a human subject.

21. The method of claim 20, wherein the human subject has a known genotype at a genetic locus.

22. The method of claim 20, wherein the human subject is suspected of or known to have a disorder.

23. The method of claim 22, wherein the disorder is an infection.

24. The method of claim 20, wherein the sample is a body fluid or a processed form of a body fluid.

25. The method of claim 18 wherein the sample comprises a test substance and wherein a test substance that causes a change in the output signal is an agonist or antagonist of the receptor.

26. A method for measuring the change in activation state of a multi-ligand-responsive receptor in response to a test condition comprising:
measuring the output signal produced by an interactive sensor pair in a control condition, the interactive sensor pair comprising a first polypeptide and a second polypeptide, wherein the first polypeptide comprises a multiple-ligand-responsive receptor, and wherein the second polypeptide comprises a polypeptide that binds to the first polypeptide, the binding being affected by binding of a ligand to the first polypeptide, and wherein the first polypeptide or the second polypeptide is stably attached to a reactive module;
exposing the interactive sensor pair to the test condition; and
measuring the output signal;
wherein a change in output signal from the control condition to the test condition indicates a change in activation state of the receptor in response to the test condition.

27. The method of claim 26, wherein the first polypeptide is stably attached to a reactive module and the second polypeptide is stably attached to a reactive module.

28. A method for determining the effect of an allelic pattern on a biological activity in a subject, comprising:
detecting an allelic pattern in a nucleic acid sample obtained from the subject;
contacting a biological sample obtained from the subject with a detection reagent; and
measuring the output signal,
wherein the output signal integrates the effects of the allelic pattern on the biological activity in the subject.

29. The method of claim 28, wherein the detection reagent comprises a first polypeptide and a second polypeptide, and wherein the first polypeptide comprises an IL-1R1-like receptor.

30. A method of claim 28, wherein the allelic pattern is an IL-1 allelic pattern.

31. A method for generating a database system for integrating genetic and non-genetic information, comprising:
detecting an allelic pattern in nucleic acid samples obtained from a plurality of subjects;
contacting biological samples obtained from the plurality of subjects with a detection reagent;
measuring the output signals produced by the interactive sensor pair in response to each biological sample;
entering into a database a record for each allelic pattern detected; and
entering into a database a record for each output signal detected,
wherein, for each subject, the record for the allelic pattern detected is linked to the record for the output signal detected.

32. A method of claim 31, further comprising:
obtaining clinical status information from the plurality of subjects;
entering into a database a record for the clinical information obtained,
wherein, for each subject, the record for the clinical status information is linked to the record for the allelic pattern detected and the record for the output signal detected.

33. A computer system comprising a database generated by the method of claim 31 and a user interface allowing a user to selectively view information regarding allelic patterns and output signals.

34. A method for selecting an appropriate targeted therapeutic for a subject, comprising:
contacting a biological sample obtained from the subject with a detection reagent; and
measuring the output signal,
wherein the output signal indicates the presence or absence of an abnormal biological activity associated with a disorder, and wherein a targeted therapeutic is selected to compensate for the disorder.

35. The method of claim 34, wherein the targeted therapeutic is selected to compensate for the abnormal biological activity.

36. The method of claim 34, wherein the abnormal biological activity is an abnormal assembly state of an IL-1R-like receptor.

37. The method of claim 34, the method further comprising:
detecting an allelic pattern in a nucleic acid sample obtained from the subject.

38. The method of claim 37, wherein the allelic pattern is an IL-1 allelic pattern, the interactive sensor pair comprises an IL-1R1 and the targeted therapeutic comprises an IL-1 antagonist.

39. The method of claim 38, wherein the IL-1 antagonist comprises an active ingredient selected from the group consisting of: IL-1Ra protein, an IL-1-targeted monoclonal antibody and an ICE inhibitor.

40. A recombinant fusion protein comprising:
an amino acid sequence that facilitates purification;
an IL-1R1-like amino acid sequence or an IL-1Rac sequence; and
a reactive module polypeptide.

41. The recombinant nucleic acid of claim 40, wherein the amino acid sequence that facilitates purification is a histidine tag, and wherein the IL-1R1-like amino acid sequence is the amino acid sequence of SEQ ID NO:12, or an active portion thereof, and wherein the reactive module polypeptide is a fluorescent protein.

42. The recombinant nucleic acid of claim 40, wherein the amino acid sequence that facilitates purification is a histidine tag, and wherein the IL-1Rac amino acid sequence is the amino acid sequence of SEQ ID NO:10, or an active portion thereof, and wherein the reactive module polypeptide is a fluorescent protein.

43. A nucleic acid encoding the fusion protein of claim 40.
